SEQUENCE LISTING (8 sequences)

SEQ ID NO: 1 - DNA, 21 bp, Artificial Sequence
  Synthetic polynucleotide
actcctacgggaggcagcagt

SEQ ID NO: 2 - DNA, 18 bp, Artificial Sequence
  Synthetic polynucleotide
attaccgcggctgctggc

SEQ ID NO: 3 - DNA, 22 bp, Artificial Sequence
  Synthetic polynucleotide
cttggtcatttagaggaagtaa

SEQ ID NO: 4 - DNA, 20 bp, Artificial Sequence
  Synthetic polynucleotide
gctgcgttcttcatcgatgc

SEQ ID NO: 5 - DNA, 20 bp, Artificial Sequence
  Synthetic polynucleotide
attggagggcaagtctggtg

SEQ ID NO: 6 - DNA, 20 bp, Artificial Sequence
  Synthetic polynucleotide
ccgatccctagtcggcatag

SEQ ID NO: 7 - DNA, 25 bp, Artificial Sequence
  Synthetic polynucleotide
ccaaagtgggatagaaatggtagtc

SEQ ID NO: 8 - DNA, 25 bp, Artificial Sequence
  Synthetic polynucleotide
tcactcatcctcattcacaaagact

(19) United States
(12) Patent Application Publication
(10) Pub. No.: US 2019/0183950 A1
(43) Pub. Date: Jun. 20, 2019

(54) COMMENSAL FUNGI AND COMPONENTS THEREOF FOR USE IN MODULATING IMMUNE RESPONSES

(71) Applicant: Children's Hospital Medical Center, Cincinnati, OH (US)

(72) Inventor: Sing Sing Way, Cincinnati, OH (US)

(73) Assignee: Children's Hospital Medical Center, Cincinnati, OH (US)

(21) Appl. No.: 16/226,254

(22) Filed: Dec. 19, 2018

Related U.S. Application Data
(60) Provisional application No. 62/608,116, filed on Dec. 20, 2017.

(51) Int. Cl. A61K 36/062; A61K 36/064; A61P 37/02; A61P 31/10; A61K 45/06

(52) U.S. Cl. CPC A61K 36/062; A61K 36/064; A61K 45/06; A61P 31/10; A61P 37/02

(57) ABSTRACT

Compositions and methods of modulating immune responses (e.g., immune responses mediated by Th17 CD4⁺ T cells), involving fungal cells (Candida cells or yeast cells), extracts thereof comprising mannan molecules, anti-fungal agents, and/or mycotic probiotics.

### CROSS-REFERENCE TO RELATED APPLICATIONS

This application claims the benefit under 35 U.S.C. § 119(e) of U.S. provisional application No. 62/608,116, filed Dec. 20, 2017, the contents of which are incorporated by reference herein in their entirety.

### GOVERNMENT RIGHTS

This invention was made with government support under NIH R21-AI123089 and NIH R21-AI128932, awarded by the National Institutes of Health. The government has certain rights in the invention.

### BACKGROUND OF THE INVENTION

Commensal intestinal microbes collectively play beneficial roles in calibrating immunological responsiveness to improve the outcomes of inflammatory disorders and infections (Chung and Kasper, Current opinion in immunology 22:455-460 (2010); Hooper et al., Science 336:1268-1273 (2012); Round and Mazmanian, Nat Rev Immunol 9:313-323 (2009)). This symbiotic relationship between enteric commensal microbes and the mammalian host has primarily been probed by analyzing intestinal bacteria. For example, oral administration of broad-spectrum antibiotics that deplete enteric bacteria impairs resiliency and survival against dextran sodium sulfate (DSS)-induced colitis (Rakoff-Nahoum et al., Cell 118:229-241 (2004)). Interestingly, these protective benefits are not confined to local intestinal tissues, but extend distally to enhance responsiveness of immune cells that protect against pathogens that disseminate or replicate in extra-intestinal tissues (Abt et al., Immunity 37:158-170 (2012); Ichinohe et al., Proc Nati Acad Sci USA 108:5354-5359 (2011)). These collective benefits of commensal bacteria are reproduced by intestinal stimulation using conserved bacterial structural components such as peptidoglycan or LPS (Abt et al., Immunity 37:158-170 (2012); Clarke et al., Nat Med 16:228-231 (2010); Ichinohe et al., Proc Natl Acad Sci USA 108:5354-5359 (2011); Rakoff-Nahoum et al., Cell 118:229-241 (2004)). Thus, commensal bacteria, through their principal molecular components, play important immune-modulatory roles in protecting against disease locally in the intestine and systemically in extra-intestinal tissues.

Importantly however, microbial commensalism is not restricted only to bacteria, but includes viral and fungal species each capable of colonizing mammalian hosts. This overlap between commensal bacteria and viruses gives rise to fundamental questions regarding whether other classes of intestinal microbes play similar beneficial roles in positively calibrating local and systemic immunity.

Fungi are a ubiquitous component of the mammalian microbiome. Despite being estimated to comprise <1% of commensal microbial species by genomic equivalence (Arumugam et al., Nature 473:174-180 (2011); Qin et al., Nature 464:59-65 (2010)), individual fungi are also of >100-fold increased size compared with bacteria (Underhill and Lliev, Nature Reviews Immunology 14:405-416 (2014)). Thus, whether fungal-host interactions are autonomously beneficial for influencing disease outcomes remains undefined.

### SUMMARY OF THE INVENTION

The present disclosure is based, at least in part, on the discoveries that commensal fungi (e.g., C. albicans or S. cerevisiae) or cell wall constitutes thereof (containing mannans) unexpectedly overturned susceptibility to intestinal injury and extra-intestinal infection caused by absent bacteria and that commensal fungi such as C. albicans calibrated systemic Th17 immunological responses.

Accordingly, one aspect of the present disclosure features a method for modulating immune responses in a subject, the method comprising administering to a subject in need thereof an effective amount of a composition comprising fungal cells or an extract thereof that contains one or more mannans.

In some embodiments, the fungal cells (e.g., Candida albicans cells or Saccharomyces cerevisiae cells) are used in the method described herein. The fungal cells may be administered to the subject orally or intrarectally. When delivered to a subject, the fungal cells may colonize in the intestine of the subject after administration. In other embodiments, the extract of fungal cells can be used in the method described herein.

In any of the methods described herein, the subject can be a human subject. In some examples, the human subject may have or may be at risk for intestinal mucosal tissue injury, intestinal infection, or imbalanced intestinal microbiota. In some instances, the infection may be caused by a bacterium such as Streptococcous pneumoniae, Klebsiella pneumoniaie, Escherichia coli, or a fungus such as Candida or Aspergillus. Alternatively or in addition, the subject is on treatment of an antibiotic.

In another aspect, the present disclosure features a method for modulating immune responses mediated by T helper 17 cells, the method comprising administering to a subject in need thereof (a) fungal cells or (b) an anti-fungus agent or a mycotic probiotic.

In some embodiments, the subject is administered the fungal cells, for example, Candida albicans cells, which may colonize in the intestine of the subject. In some examples, the subject is a human subject, e.g., a human subject having or at risk for microbial infection, for example infections caused by or at risk of being caused by various pathogens, for example, by a bacterium such as Streptococcous pneumoniae, Klebsiella pneumoniaie, Escherichia coli, or a fungus such as Candida or Aspergillus. In some examples, the fungal cells can be administered orally or intrarectally.

In some embodiments, the subject can be administered with an anti-fungal agent or a mycotic probiotic. Such a subject can be a human subject, for example, a human subject having or at risk for an allergic airway disorder, an autoimmune disorder (e.g., rheumatoid arthritis, lupus, or multiple sclerosis), or an autoinflammatory disorder (e.g., inflammatory bowel disease, psoriasis, or esophagitis). Exemplary anti-fungal agents include, but are not limited to, a polyene antifungal agent, an azole antifungal agent, an allylamine and morpholine antifungal agent, or an antimetabolite antifungal agent. In some instances the anti-fungus agent is administered to the subject orally.

Also within the scope of the present disclosure are commensal fungal cells or mannan-containing components thereof for use in modulating immune response, alleviating intestinal infection and/or intestinal injury, or uses of commensal fungal cells or mannan-containing components thereof for manufacturing medicaments for the intended medical uses as described herein.

The details of one or more embodiments of the invention are set forth in the description below. Other features or advantages of the present invention will be apparent from the following drawings and detailed description of several embodiments, and also from the appended claims.

### BRIEF DESCRIPTION OF THE DRAWINGS

The following drawings form part of the present specification and are included to further demonstrate certain aspects of the instant application, which can be better understood by reference to one or more of these drawings in combination with the detailed description of specific embodiments presented herein.

FIGS. 1A-1H include graphs showing Candida albicans intestinal mono-colonization bypasses the protective necessity of commensal enteric bacteria. 1A: graphs showing recoverable bacterial colony forming units (CFUs) from feces of mice after supplementing the drinking water with an antibiotic cocktail (ABX) containing ampicillin, gentamicin, metronidazole, neomycin, and vancomycin (left panel), compared with no-antibiotic treated conventional (CNV) controls housed under specific-pathogen free conditions (right panel). 1B: a graph showing bacterial-specific 16S rDNA qPCR of feces for mice described above normalized to conventional (CNV) controls housed under specific-pathogen free conditions. 1C: a graphical representation of the dosing time points and a graph of recoverable fungal CFUs in the feces (middle panel) and each intestinal segment (right panel) for mice inoculated with C. albicans (CA) and maintained on ABX treatment for 14 days (ABX+CA), compared to ABX treated controls without CA administration (ABX). The left panel illustrates treatment schedule of the mice. 1D: graphs showing fungal-specific internal transcribed spacer (ITS-1) rDNA (left) and eukaryotic 18S rDNA (right) qPCR of feces for mice described above normalized to germ-free (GF) controls. 1E: a graph showing weight gain after C. albicans inoculation among antibiotic treated mice (ABX+CA) compared with ABX only controls. 1F: graphs showing percent survival (right panel) after DSS supplementation in the drinking water (for six days, shown in left panel) for mice described above. 1G: graphs showing percent survival after influenza A PR8-OVA (Flu A) intranasal infection (6×10⁴ PFUs) for mice described above. Left panel illustrates treatment schedule and right panel shows survival rates. 1H: graphs showing total number of Flu A-specific KbOVA tetramer+ CD8 T cells (left), and IFN-γ+ CD8 T cells after in vitro OVA257-264 peptide stimulation (right), from lungs nine days after Flu A infection for mice described in FIG. 1A, FIG. 1B, and FIG. 1C. *P<0.05, **P<0.01, ***P<0.001, ****P<0.0001, by Log-rank (Mantel-Cox) test (F,G) or non-parametric Kruskal-Wallis test with Dunn's correction (H). Data are representative of at least two independent experiments each with similar results. Bars, mean±s.e.m. L.o.D., limit of detection.

FIGS. 2A-2E include diagrams showing that C. albicans enteric colonization does not cause intestinal tissue injury. 2A-2C: Composite data enumerating villus width (2A), goblet cells per villus (2B), and granules per paneth cell (2C), for specific-pathogen free mice administered C. albicans and maintained on antibiotics containing ampicillin, gentamicin, metronidazole, neomycin, vancomycin for 14 days (ABX+CA), compared with antibiotic treated controls without C. albicans inoculation (ABX). 2D-2E: Composite data enumerating crypt height (2D), and number of goblet cells per 40× field (2E) for mice described in panels 2A-2C. Data are representative of at least two independent experiments. Bars: mean±s.e.m.

FIGS. 3A-3D include graphs showing C. albicans (CA) protects against DSS induced colonic shortening, intestinal permeability and inflammation compared with antibiotic treated control mice. 3A: a diagram showing treatment schedule. 3B: a chart showing colon length of mice treated with ABX or ABX in combination with CA. 3C: a chart showing dextran level in mice treated with ABX or ABX in combination with CA. 3D: graphs showing disease states (e.g., reflected by epithelial ulceration, inflammatory cell infiltration or edema) of mice treated with ABX or ABX+CA. Total panel is a photo showing H&E staining of intestinal tissue samples from mice treated with ABX or ABX+CA. Bottom panels are charts showing disease states.

FIGS. 4A-4B include diagrams showing C. albicans mono-association significantly improved survival of germ-free mice. 4A: a chart showing levels of fungal cells in germ-free (GF) mice and GF mice treated with CA. 4B: a chart showing survival rate of GF mice treated with DSS and GF mice treated with DSS and CA.

FIGS. 5A-5E are graphs showing Saccharomyces cerevisiae intestinal mono-colonization overturns disease susceptibility induced by commensal bacteria depletion. 5A: graphs showing recoverable fungal colony forming units (CFUs) in the feces of specific-pathogen free mice administered S. cerevisiae (SC) (right panel) and maintained on drinking water supplemented with an antibiotic cocktail containing ampicillin, gentamicin, metronidazole, and neomycin, vancomycin for 14 days (ABX+CA; left panel), compared with antibiotic treated controls without SC inoculation (ABX). 5B: a graph showing percent survival after DSS supplementation in the drinking water (for six days) for mice described above. 5C: a graph showing colon length after DSS treatment (for six days) for mice described above. 5D: a graph showing percent survival after influenza A PR8-OVA (Flu A) intranasal infection (6×10⁴ PFUs) for mice described above. Left panel illustrates a treatment schedule. Right panel shows survival rates. 5E: graphs showing the total number of Flu A-specific KbOVA tetramer+ CD8 T cells (left), and IFN-γ+ CD8 T cells after in vitro OVA257-264 peptide stimulation (right), from lungs nine days after Flu A infection for mice described above.

FIGS. 6A-6C are graphs showing persistent fungal intestinal colonization is required for maintaining their protective benefits. 6A: graphs showing fungal colony forming units (CFUs) in the feces with or without supplementing fluconazole (FLUC) to specific-pathogen free mice previously administered C. albicans (CA) and maintained on drinking water supplemented with an antibiotic cocktail (ABX) containing ampicillin, gentamicin, metronidazole, neomycin, and vancomycin. Left panel illustrates a treatment schedule. Right panel shows fungal CFU values. 6B: graphs showing percent survival after DSS supplementation in the drinking water (for six days) for mice described above. Left panel illustrates a treatment schedule. Right panel shows survival rates. 6C: graphs showing percent survival after influenza A PR8-OVA (Flu A) intranasal infection (6×10⁴ PFUs) for mice described above. Left panel illustrates a treatment schedule. Right panel shows survival rates.

FIGS. 7A-7D are graphs showing the protective benefits of commensal fungi are mediated by mannans and through TLR4 dependent pathways. 7A: graphs showing percent survival after DSS supplementation (for six days) among specific-pathogen free mice maintained on drinking water supplemented with an antibiotic cocktail (ABX) containing ampicillin, gentamicin, metronidazole, neomycin, and vancomycin, and administered C. albicans (CA) three days after initiating antibiotic treatment, or intrarectally administered mannan, curdlan, zymosan or saline (PBS) every other day starting one day prior to DSS challenge. Left panel illustrates a treatment schedule. Right panel shows survival rates. 7B: a graph showing NF-kB expression induced among RAW-blue macrophages after incubation with or without mannan in the presence or absence of the indicated neutralizing antibodies. 7C: a graph showing percent survival after DSS supplementation (for six days) among conventional (CNV) dectin-1 deficient mice housed in specific-pathogen free conditions, administered antibiotics (ABX), or C. albicans (CA) inoculated three days after initiating antibiotic treatment (ABX+CA). Left panel illustrates a treatment schedule. Right panel shows survival rates. 7D: a graph showing percent survival after DSS supplementation (for six days) among conventional (CNV) TLR4 deficient mice housed in specific-pathogen free conditions, administered antibiotics (ABX), or C. albicans (CA) inoculated three days after initiating antibiotic treatment (ABX+CA). Left panel illustrates a treatment schedule. Right panel shows survival rates.

FIGS. 8A-8B include diagrams showing mannan reconstitution enhanced survival and accumulation of protective immune cells in mice treated with DSS or influenza A virus. 8A: graphs showing mannan intrarectal reconstitution or treatment with C. albicans (CA) enhanced survival of mice infected with influenza A virus. Left panel illustrates a treatment schedule. Right panel shows survival rates. 8B: charts showing accumulation of K^{b}OVA⁺ CD8 T cells (left panel) and IFNγ⁺ CD8 T cells in mice treated with CA or mannan.

FIG. 9A-9F include graphs showing that C. albicans intestinal colonization protects against systemic C. albicans invasive infection. 9A: graphs showing recoverable C. albicans colony forming units (CFUs) from the feces of mice with ampicillin supplementation in the drinking water compared with no antibiotic controls after oral C. albicans inoculation. Left panel illustrates a treatment schedule. Right panel shows fungal CFU values. 9B: a graph showing recoverable C. albicans CFUs in each intestinal segment seven day after oral C. albicans inoculation for the mice described above. 9C: a graph showing recoverable C. albicans CFUs in each tissue seven day after oral C. albicans inoculation for mice supplemented with ampicillin in the drinking water. 9D: a graph showing weight change after C. albicans oral inoculation for the mice described above. 9E: graphs depicting percent survival and recoverable C. albicans CFUs from the kidneys five days after C. albicans intravenous infection (5×10⁴ CFUs) for mice with C. albicans intestinal colonization or no colonization control mice maintained on ampicillin supplemented drinking water. Left panel illustrates a treatment schedule. Middle panel shows survival rates. Right panel shows CFU values. 9F: a photo showing fluorescence intensity for C. albicans recovered from the feces compared with the kidneys five days after intravenous infection with GFP+ C. albicans for mice with prior oral inoculation with GFP− C. albicans.

FIGS. 10A-10E include graphs and data showing systemic expansion of protective IL-17 producing CD4⁺ T cells with commensal C. albicans specificity. 10A: graphs of representative plots (left panel) and composite data (middle and right panels) showing total number and percent CD44^{hi} among I-Ab:2W1S tetramer positive CD4+ T cells from spleen and peripheral lymph nodes of mice with C. albicans intestinal colonization compared with no colonization controls. 10B: Representative plots (left panel) and composite data (middle and right panels) showing percent and total number RORγt+ among I-Ab:2W1S positive (solid line) or negative (gray shaded) CD4+ T cells for each group of mice described above. 10C: graphs of representative plots (left panel) and composite data (right panels) showing percent IL-17A, IL-17F or IFN-γ production by CD4+ splenocyte and lymph nodes cells after heat-killed C. albicans stimulation for each group of mice described above. 10D: a graph showing recoverable C. albicans CFUs from the kidneys five days after C. albicans intravenous infection (5×10⁴ CFUs) for C. albicans colonized compared with or no colonization control mice administered rat IgG (isotype), anti-CD4, or anti-IL-17A plus anti-IL-17F antibodies one day prior to infection. 10E: a graph showing percent of survival for the mice described above.

FIGS. 11A-11C include graphs of representative plots and composite data showing percent RORγt+ (10A), T-bet+ (10B) and FOXP3+ (10C) among CD4+ T cells with I-Ab:2W1S specificity (solid line) or compared with bulk CD4+ T cells (gray shaded) for cells from the spleen and peripheral lymph nodes 10 days after intestinal colonization with 2W1S-expressing C. albicans compared with no colonization controls.

FIGS. 12A-12F are graphs showing C. albicans intestinal colonization stimulates accumulation, activation and IL-17 responsiveness by circulating neutrophils. 12A: graph showing representative plots (left panel) and composite data (right panel) showing percent IL-17RC+ amongst CD45+ leukocytes in the peripheral blood of mice with C. albicans intestinal colonization compared with no colonization controls. 12B: a graph showing percent of IL-17RC+ amongst each leukocyte subsets for each group of mice described above. 12C: a graph showing percent IL-17RC+ amongst CD45+ leukocytes in the peripheral blood of C. albicans colonized mice 7 days after the administration of rat IgG (isotype), anti-CD4, or anti-IL-17A plus anti-IL-17F antibodies. 12D: a graph showing percent of Ly6G+Ly6Cint neutrophils amongst CD45+ leukocytes in the peripheral blood for each group of mice described above. 12E: a graph showing representative plots (solid line histogram, C. albicans stimulation; shaded histogram, no stimulation controls) and composite data showing the relative proportion of dihydrorhodamine (DHR)123 fluorescence amongst Ly6G+Ly6Cint neutrophils in the peripheral blood for each group of mice described above. 12F: graphs depicting percent survival after C. albicans intravenous infection (5×10⁴ CFUs) amongst C. albicans colonized compared with or no colonization control mice administered anti-Ly6G (left panel) or anti-Gr1 (right panel) antibodies one day prior to infection.

FIGS. 13A-13B are graphs showing fluconazole eradicates C. albicans intestinal colonization. 13A: graphs of recoverable C. albicans colony forming units (CFUs) from the feces of mice each day after initiating fluconazole to the ampicillin supplemented drinking water. Left panel illustrates a treatment schedule. Right panel shows fungal CFU values. 13B: graphs of recoverable C. albicans CFUs from the kidneys five days after C. albicans intravenous infection (5×10⁴ CFUs) at each time point after discontinuation of fluconazole drinking water supplementation, compared with mice with sustained fluconazole drinking water supplementation or no fluconazole treatment controls. Left panel illustrates a treatment schedule. Right panel shows CFU values.

FIGS. 14A-14G include graphs showing protection against systemic C. albicans invasive infection requires persistent C. albicans intestinal colonization. 14A: graphs showing recoverable C. albicans CFUs from the kidneys five days after C. albicans intravenous infection (5×10⁴ CFUs) for C. albicans colonized mice treated with fluconazole, no antifungal treatment controls with sustained C. albicans intestinal colonization, or no C. albicans colonization control mice. Left panel illustrates a treatment schedule. Right panel shows CFU values. 14B: shows representative plots (left panel) and composite data (right panel) showing total number of I-Ab:2W1S tetramer positive CD4+ T cells from spleen and peripheral lymph nodes for C. albicans colonized mice treated with fluconazole for 20 days, no antifungal treatment controls with sustained C. albicans intestinal colonization, or no C. albicans colonization control mice. 14C: graphs showing percent (left panel) and total number (right panel) RORγt+ among I-Ab:2W1S positive CD4+ T cells for each group of mice described above. 14D: shows representative plots (left panel) and composite data (right panel) showing percent IL-17A or IL-17F production by CD4+ splenocyte and lymph nodes cells after heat-killed C. albicans stimulation for each group of mice described above. 14E: graphs showing percent IL-17RC+ amongst CD45+ leukocytes (left panel) or Ly6G+Ly6Cint neutrophils (right panel) in the peripheral blood of mice described above. 14F: a graph showing percent of Ly6G+Ly6Cint neutrophils amongst CD45+ leukocytes in the peripheral blood for each group of mice described above. 14G: a graph showing relative intensity of dihydrorhodamine (DHR)123 fluorescence amongst Ly6G+Ly6Cint neutrophils in the peripheral blood for each group of mice described above.

FIGS. 15A-15C are graphs showing commensal C. albicans protects against S. aureus infection and promotes susceptibility to Th17 airway inflammation. 15A: graphs showing percent survival and recoverable S. aureus CFUs from the kidneys five days after S. aureus intravenous infection (strain USA300; 108 CFUs [middle panel] or 3×10⁷ CFUs [right panel]) for mice with C. albicans intestinal colonization or no colonization control mice. Left panel illustrates a treatment schedule. 15B: graphs showing percent survival (middle panel) and infection clinical score tempo (right panel) after influenza A virus intranasal infection (strain PR8, 2×10⁵ PFU) for mice with C. albicans intestinal colonization or no colonization control mice. Left panel illustrates a treatment schedule. 15C: graphs showing Airway resistance (right bottom), percent RORγt+ (left bottom) or IL-17A production by CD4+ T cells (middle bottom) recovered from the lungs of C. albicans colonized mice treated with fluconazole, no antifungal treatment controls with sustained C. albicans intestinal colonization, or no C. albicans colonization control mice. Mice were intratracheally sensitized and challenged with house dust mite extract, and airway resistance represent change over baseline after inhaled methacholine challenge (top).

FIG. 16A-16B include graphs showing C. albicans fecal colonization density positively correlates with systemic levels of fungal-specific Th17 inflammation. 16A: graphs showing representative diagrams and plots (left panel and right panel) showing IL-17A or IL-17F production by CD4+ T cells amongst peripheral blood mononuclear cells of after heat-killed C. albicans plus anti-CD28 stimulation compared with anti-CD28 stimulation controls, and regression analysis of this data compared with the density of C. albicans in the feces of each individual. 16B: a graph showing regression analysis comparing the density of C. albicans in the feces and intensity of IL-17RC staining by CD15+CD16+ neutrophils amongst peripheral blood mononuclear for each individual.

### DETAILED DESCRIPTION OF THE INVENTION

Commensal intestinal microbes collectively play beneficial roles in calibrating immunological responsiveness to improve the outcomes of inflammatory disorders and infections (Chung and Kasper, Current opinion in immunology 22:455-460 (2010); Hooper et al., Science 336:1268-1273 (2012); Round and Mazmanian, Nat Rev Immunol 9:313-323 (2009)). However, studies on the symbiotic relationship between enteric commensal microbes and the mammalian host have primarily focused on intestinal bacteria. Besides bacteria, intestinal microbes also include fungi and virus. There is limited information as to the roles of such intestinal fungi and virus mammalian host.

The present disclosure provides unexpected discoveries that commensal fungi (e.g., C. albicans and S. cerevisiae), when colonized in the intestine, overturned disease susceptibility induced by depletion of commensal bacteria and showed protective effects against infection and tissue damage. Fungi extract containing mannans showed similar protective effects. Further, it was discovered that commensal fungi such as C. albicans modulate immune responses systemically (e.g., systemic immune responses mediated by Th17 CD4⁺ T cells). For example, C. albicans intestinal colonization was found to protect against invasive C. albicans infection and induce systemic Th17 CD4⁺ cell differentiation, leading to tonic neutrophil activation to improve host defense against extracellular pathogens. On the other hand, increased susceptibility to IL-17 driven allergic airway inflammation may occur with intestinal C. albicans colonization, suggesting that anti-fungal agents and/or mycotic probiotics may be effective in tuning systemic immunity, thereby benefiting the treatment of diseases associated with elevated Th17 CD4⁺ T cells and IL-17.

Accordingly, provided herein are pharmaceutical compositions and methods of modulating immune responses using commensal fungi cells, mannan-containing extracts thereof, or anti-fungal agents and/or mycotic probiotics in subjects in need of the treatment.

### Commensal Fungi and Mannan-Containing Extracts Thereof

Commensal fungi refers to fungi cells capable of living with other microorganisms in a host (e.g., a mammalian host such as a human host) in a relationship, in which one microorganism derives food or other benefits from another microorganism without hurting or helping it.

In some embodiments, the commensal fungal cells for use in the methods described herein can be a fungal species naturally existing in a host such as a human host. Such fungal species can be identified by analyzing the mycobiota in a host of interest using conventional methodology, e.g., restriction fragment length polymorphism (RFLP) analysis, oligonucleotide fingerprinting of rRNA genes (OFRG), or denaturing gradient gel electrophoresis (DGGE). Specific fungal species can be identified by, e.g., analyzing fungal rDNA sequences such as 18S rDNA sequences and comparing the resultant sequences with fungal rDNA databases to determine identify of the fungal species. See, e.g., Underhill et al., Nat. Rev. Immunol. 14(6):405-416 (2014).

In some embodiments, the fungal cells for use in the methods described herein can be of Aspergillus, Aureobasidium, Arthrodermatoceae, Alternaria, Candida, Cladosporium, Cryptococcus, Cystofilobasidium, Debaryomyces, Epicoccum, Fusarium, Malassezia, Rhodotorula, Saccharomyces, or a combination thereof.

In some examples, the fungal cells are of Candida, Saccharomyces, or a combination thereof. Exemplary Candida species include, but are not limited to, C. tropicalis, C. albicans, C. glabrata, C. dubliniensis, C. parapsilosis, and C. rugosa. Exemplary Saccharomyces species include, but are not limited to, S. arboricolus, S. bayanus, S. boulardii, S. bulderi, S. cariocanus, S. cariocus, S. cerevisiae, S. chevalieri, S. dairenensis, S. ellipsoideus, S. eubayanus, S. exiguous, S. florentinus, S. fragilis, S. kluyveri, S. kudriavzevii, S. martiniae, S. mikatae, S. monacensis, S. norbensis, S. paradoxus, S. pastorianus, S. spencerorum, S. turicensis, S. unisporus, S. uvarum, or S. zonatus.

In some embodiments, a composition comprising one or more mannan molecules, such as an extract of fungal cells that comprising one or more mannan molecules, can be used in the method described herein. Mannan molecules are a ground of plant polysaccharides (e.g., a linear polymer of mannose, which may be in β1,4-linkage). Mannans are a major cell wall component shared by nearly all fungi (Klis et al., Yeast 23:185-202 (2006); Ruiz-Herrera et al., FEMS Yeast Res 6:14-29 (2006)). In some examples, the mannan molecules may be synthesized chemically. In other examples, the mannan molecules may be extracted from a suitable fungal species, e.g., C. albicans or S. cerevisiae. An extract of fungal cells refer to a composition of fungal cell components extracted from fungal cells by any suitable means, for example, solvent extraction, fragmentation, etc.

### Anti-Fungal Agents and Mycotic Probiotics

Anti-fungal agents are molecules or combinations thereof that show bioactivities in inhibiting fungal cell growth and/or killing fungal cells. Examples include polyene anti-fungal compounds (e.g., amphotericin, nystatin, and pimaricin), azole anti-fungal compounds (e.g., fluconazole, itraconazole, and ketoconazole), allylamine anti-fungal compounds (e.g., allylamines such as naftifine and terbinafine), morpholine anti-fungal compounds (e.g., amorolfine), and antimetabolite anti-fungal compounds (e.g., f-Fluorocytosine). Specific examples include (but are not limited to) clotrimazole, econazole, miconazole, terbinafine, fluconazole, ketoconazole, and amphotericin.

Mycotic probiotics refer to probiotics that possess anti-fungal activities. Probiotics comprises live bacteria that confer beneficial effects to the host organism when colonized in the gut of a host organism. For example, they metabolize a food ingredient that is non-digestible to the host organism, modulate the host immune system in a non-pathogenic manner, prevent growth of pathogenic bacteria, and/or produce nutrients that can be taken up by the host (e.g., biotin and vitamin K). Beneficial bacteria include, but are not limited to bifidobacteria, lactobacilli, Bacteroides fragilis, Bacteroides thetaiotaomicron, Enterococcus faecalis, Staphylococcus epidermides, Enterobacter aerogenes, and Enterobacter cloacae. The probiotics for use in the method described herein can be a population of bifidobacteria, lactobacilli, Bacteroides fragilis, Bacteroides thetaiotaomicron, Enterococcus faecalis, Staphylococcus epidermides, Enterobacter aerogenes, Enterobacter cloacae, or related bacteria having similar functions.

### Pharmaceutical Compositions

In some embodiments, the fungal cells, extracts thereof, mannan molecules, anti-fungal agent, or mycotic probiotics described herein can be formulated for administration to a subject as a pharmaceutical composition, e.g., together with a pharmaceutically acceptable carrier, diluent or excipient.

A carrier, diluent or excipient that is “pharmaceutically acceptable” includes one that is sterile and pyrogen free. Suitable pharmaceutical carriers, diluents and excipients are well known in the art. The carrier(s) must be “acceptable” in the sense of being compatible with the inhibitor and not deleterious to the recipients thereof.

A pharmaceutical composition comprising any of the fungal cells, extracts thereof, mannan molecules, anti-fungal agent, or mycotic probiotics described herein may be administered by any administration route known in the art, such as parenteral administration, oral administration, buccal administration, sublingual administration, topical administration, or inhalation, in the form of a pharmaceutical formulation comprising the active ingredient, optionally in the form of a non-toxic organic, or inorganic, acid, or base, addition salt, in a pharmaceutically acceptable dosage form. In some embodiments, the administration route is oral administration and the formulation is formulated for oral administration. In other embodiments, the administration route is rectal (e.g., intrarectal) and the composition is formulated for rectal administration.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. Aqueous solutions may be suitably buffered (preferably to a pH of from 3 to 9). The preparation of suitable parenteral formulations under sterile conditions is readily accomplished by standard pharmaceutical techniques well-known to those skilled in the art.

In some embodiments, the pharmaceutical composition or formulation is suitable for oral, buccal or sublingual administration, such as in the form of tablets, capsules, ovules, elixirs, solutions or suspensions, which may contain flavoring or coloring agents, for immediate-, delayed- or controlled-release applications.

Suitable tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycolate, croscarmellose sodium and certain complex silicates, and granulation binders such as polyvinylpyrrolidone, hydroxypropylmethylcellulose (HPMC), hydroxy-propylcellulose (HPC), sucrose, gelatin and acacia. Additionally, lubricating agents such as magnesium stearate, stearic acid, glyceryl behenate and talc may be included.

Solid compositions of a similar type may also be employed as fillers in gelatin capsules. Preferred excipients in this regard include lactose, starch, a cellulose, milk sugar or high molecular weight polyethylene glycols. For aqueous suspensions and/or elixirs, the compounds of the invention may be combined with various sweetening or flavouring agents, coloring matter or dyes, with emulsifying and/or suspending agents and with diluents such as water, ethanol, propylene glycol and glycerin, and combinations thereof.

The formulations may be presented in unit-dose or multi-dose containers, for example sealed ampoules or vials, and may be stored in a freeze-dried (lyophilized) condition requiring only the addition of the sterile liquid carrier immediately prior to use.

In some embodiments, any of the agent described herein can be administered to a subject at a dose of between 0.1 to 1,000 mg per subject or between 0.001 to 10 mg/kg per subject, administered in single or divided doses. A physician in any event may determine the actual dosage which will be most suitable for any subject, which will vary with the age, weight and the particular disease or disorder to be treated or prevented.

### Therapeutic Applications

Aspects of the disclosure relate to a method of modulating immune responses, for example, immune responses mediated by TH17 CD4⁺ T cells, using the fungal cells, extracts thereof, mannan molecules, anti-fungal agent, or mycotic probiotics as described herein. To perform such a method, an effective amount of the fungal cells, mannan-containing extracts thereof, one or more mannan molecules, anti-fungal agents, and/or mytotic probiotics as described herein can be administered to a subject in need of the treatment via a suitable route, for example, oral administration or rectal administration. When fungal cells are used, it is expected that the fungal cells will colonize in the digestive tract (e.g., in the intestine) of the subject after administration.

An “effective amount,” or an “amount effective to”, as used herein, refers to an amount of the fungal cells, extracts thereof, mannan molecules, anti-fungal agent, or mycotic probiotics as described herein that is effective in producing the desired therapeutic, ameliorative, inhibitory or preventative effect, and/or results in a desired clinical effect, such as modulating immune responses in the subject (e.g., activate or suppress immune responses mediated by Th17 CD4⁺ T cells or IL-17), alleviating susceptibility to infection caused by pathogens such as bacteria or virus due to depletion of intestinal bacteria, and/or alleviating intestinal tissue damage in the subject.

In some embodiments, the method described herein comprises administering an effective amount of fungal cells (e.g., Candida cells such as C. albican cells or Saccharomyces cells such as S. cerevisiae cells) or a composition comprising one or more mannan molecules (e.g., an extract of fungal cells that comprises mannans) to a subject in need of the treatment. The subject may be a human subject who has or is at risk for infection, intestinal mucosal tissue injury, imbalanced intestinal microbiota, which may be caused by depletion of gut bacteria. Such a subject may be on antibiotic treatment (e.g., have undergone an antibiotic treatment or is currently treated with antibiotics). Alternatively or in addition, the subject to be treated by the method described herein may have a microbial infection, e.g., an intestinal microbial infection. Such an infection may be caused by a pathogen, for example, a virus, a bacterium (e.g., Streptococcous pneumoniae, Klebsiella pneumoniae, and Escherichia coli), or a fungus (e.g., Candida or Aspergillus).

In some embodiments, the method described herein comprises administering an effective amount of fungal cells (e.g., those described herein such as C. albicans or S. cerevisiae) to a subject in need of the treatment to stimulate immune responses mediated by Th17 CD4⁺ T cells and/or IL-17. The subject may be a human subject having or at risk for microbial infection or other conditions that would benefit from enhancing the Th17-mediated immune responses. For example, the subject may have or be at risk for an infection caused by a pathogen. In some instances, the human patient may have or be at risk for an infection caused by a bacterium. Exemplary bacterial pathogens include, but are not limited to, Streptococcous pneumoniae, Klebsiella pneumoniaie, or Escherichia coli. In other instances, the human patient may have or be at risk for an infection caused by a fungal pathogen, for example, Candida or Aspergillus.

In other embodiments, the method described herein comprises administering an effective amount of an anti-fungal agent and/or a mycotic probiotics to suppress immune responses mediated by Th17 CD4⁺ T cells and/or IL-17 in a subject in need of the treatment. The subject may be a human subject having or at risk for an allergic airway disorder (e.g., an allergic airway disorder mediated by IgE), an autoimmune disorder such as an autoinflammatory disorder. Examples include, but are not limited to, rheumatoid arthritis, lupus, multiple sclerosis, inflammatory bowel disease, psoriasis, or esophagitis. In some instances, the subject may have or be at risk for other indications that would benefit from suppression the Th17-mediated immune responses.

In some embodiments, the fungal cells, extracts thereof, mannan molecules, anti-fungal agent, or mycotic probiotics as described herein can be administered at a specific period before, during, or after a target indication (e.g., infection or depletion of gut microbiota) has occurred in the subject. In some embodiments, the fungal cells, extracts thereof, mannan molecules, anti-fungal agent, or mycotic probiotics as described herein is administered prior to manifestation of one or more symptoms of the target indication. In other embodiments, the fungal cells, extracts thereof, mannan molecules, anti-fungal agent, or mycotic probiotics as described herein are administered to the subject during or after manifestation of one or more symptoms of the target indication, or during or after occurrence of the target indication, such as within 12 or 24 hours of an infection or manifestation of one or more symptoms of the indication. In some embodiments, the fungal cells, extracts thereof, mannan molecules, anti-fungal agent, or mycotic probiotics as described herein are administered to the subject within 7 days (e.g., within 7, 6, 5, 4, 3, 2, or 1 days) after the subject is infected with a pathogen such as a bacterium or a virus, or manifests a symptom of the infection.

In some embodiments, the fungal cells, extracts thereof, mannan molecules, anti-fungal agent, or mycotic probiotics as described herein can be administered at a specific period before, during, or after an antibiotic treatment. In some embodiments, the fungal cells, extracts thereof, mannan molecules, anti-fungal agent, or mycotic probiotics as described herein is administered prior to an antibiotic treatment. In other embodiments, the fungal cells, extracts thereof, mannan molecules, anti-fungal agent, or mycotic probiotics as described herein are administered to the subject during or after the antibiotic treatment, such as within 12 or 24 hours of the antibiotic treatment. In some embodiments, the fungal cells, extracts thereof, mannan molecules, anti-fungal agent, or mycotic probiotics as described herein are administered to the subject within 6 months (e.g., within 3 months, within 2 months, within 1 month, or with 2 weeks) after the subject is treated with an antibiotic.

Infection can be detected using any method known in the art. Exemplary methods of detection include culture of blood, blood, amniotic fluid, spinal fluid or placenta to detect the pathogen, nucleic-acid based assays (e.g., PCR, reverse transcriptase-PCR, strand displacement amplification, transcription amplification, branched DNA assays, hybrid capture, ligase chain reaction, cleavase-invader, cycling probes, sequencing, microarray analysis, and melting curve analysis), serology and other immunological assays (e.g., agglutination assay, complement fixation, enzyme immunoassays, precipitation assay. Western blot) a blood smear, chromatography, mass spectrometry, gram stain, acid-fast or moderate (modified) acid-fast stains, India ink (colloidal carbon) stain, Warthin-Starry stain, Dieterle stain, Trichrome stain (Gomori-Wheatley stain), iron hematoxylin stain, or detection of the pathogen or a biomarker of the pathogen in a urine sample, a blood sample or a fecal sample (see, e.g., The Merck Manual. 19^{th} Edition. Infectious Diseases. Editor Robert S. Porter. Merck Publishing Group).

In some embodiments, the subject has one or more symptoms of infection. Exemplary symptoms of infection include fever (e.g., ranging between 38.2° C., and 41.2° C.), abdominal pain, back pain, vomiting, diarrhea, headache, myalgia, runny nose, sinus congestion, cough, itching or burning sensation, a white blood cell count from 3900 to 33,800 cells/mm3 or sore throat. Detection of these symptoms and other symptoms are within the skill of an medical practitioner.

Methods and compositions described herein are meant for any subject that has or is at risk of any of the target disorders described herein. The subject may be a mammal (e.g., mouse, dog, cat, rat, rabbit, horse, cow, pig, goat, or non-human primate) or a human.

### Kits for Modulating Immune Responses

Another aspect of the present disclosure relates to kits for use in modulating immune responses such as immune responses mediated by TH17 CD4⁺ T cells. Accordingly, in some embodiments, such a kit can comprise one or more of the active agents described herein, including the fungal cells, extracts thereof, mannan molecules, anti-fungal agent, or mycotic probiotics as described herein, or a pharmaceutical composition comprising the same, or a dietary supplement comprising the same.

In some embodiments, the kit can comprise instructions for use in accordance with any of the methods described herein. The instructions can comprise a description of administration of the fungal cells, extracts thereof, mannan molecules, anti-fungal agent, or mycotic probiotics as described herein, or a pharmaceutical composition comprising the same, for modulating immune responses and/or alleviating any of the target indications described herein. The instructions relating to the fungal cells, extracts thereof, mannan molecules, anti-fungal agent, or mycotic probiotics as described herein, or a pharmaceutical composition comprising the same, generally include information as to dosage, dosing schedule, and route of administration for the intended treatment. Such instructions may also include recommended weight-based dosages and/or age-based dosages.

Instructions supplied in the kits described herein are typically written instructions on a label or package insert (e.g., a paper sheet included in the kit), but machine-readable instructions (e.g., instructions carried on a magnetic or optical storage disk) are also acceptable. The label or package insert indicates that the composition is used for immune modulation or treatment of a target indication in subjects. In some embodiments, the label or package insert may indicate that the composition is suitable for use in specific groups of subjects, e.g., as described herein. For example, the label or package insert may indicate that the composition is suitable for use in human patients who has undergone or is on an antibiotic treatment. Instructions may be provided for practicing any of the methods described herein.

The fungal cells, extracts thereof, mannan molecules, anti-fungal agent, or mycotic probiotics as described herein, or a pharmaceutical composition comprising the same in the kit may be in suitable packaging. Suitable packaging includes, but is not limited to, vials, bottles, jars, flexible packaging (e.g., sealed Mylar or plastic bags or paper bags with a polyethylene liner), and the like. The packaging may be in unit doses, bulk packages (e.g., multi-dose packages) or sub-unit doses.

Kits may optionally provide additional components such as buffers and interpretive information. Normally, the kit comprises a container and a label or package insert(s) on or associated with the container.

### General Techniques

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook. et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.); Handbook of Experimental Immunology (D. M. Weir and C. C. Blackwell, eds.); Gene Transfer Vectors for Mammalian Cells (J. M. Miller and M. P. Calos, eds., 1987); Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds. Harwood Academic Publishers, 1995); DNA Cloning: A practical Approach, Volumes I and II (D. N. Glover ed. 1985); Nucleic Acid Hybridization (B. D. Hames & S. J. Higgins eds. (1985»; Transcription and Translation (B. D. Hames & S. J. Higgins, eds. (1984»; Animal Cell Culture (R. I. Freshney, ed. (1986»; Immobilized Cells and Enzymes (lRL Press, (1986»; and B. Perbal, A practical Guide To Molecular Cloning (1984); F. M. Ausubel et al. (eds.).

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

### Example 1: Commensal Fungi Recapitulate the Protective Benefits of Intestinal Bacteria

Commensal intestinal microbes are collectively beneficial in preventing local tissue injury and augmenting systemic antimicrobial immunity. However, given the near-exclusive focus on bacterial species in establishing these protective benefits, the contributions of other types of commensal microbes remain poorly defined. Here it is shown that commensal fungi can functionally replace intestinal bacteria, by conferring protection against injury to mucosal tissues and positively calibrating the responsiveness of circulating immune cells. Susceptibility to colitis and influenza A virus infection that occur upon commensal bacteria eradication are efficiently overturned by monocolonization with either C. albicans or S. cerevisiae. It is further shown that the protective benefits of commensal fungi are mediated by mannans, a highly conserved component of fungal cell walls, since intestinal stimulation with this moiety alone overrides disease susceptibility in mice depleted of commensal bacteria. Thus, commensal enteric fungi safeguard local and systemic immunity by providing tonic microbial stimulation that can functionally replace intestinal bacteria.

### Materials and Methods

### Mice

Age- and sex-matched wild-type, Dectin-1^{-/-} and TLR4^{-/-} mice on the C57BL/6 background were purchased from Charles River and housed under specific pathogen-free conditions. Germ-free C57BL/6 mice were maintained in gnotobiotic isolator units, exclusively fed autoclaved chow and water, and routinely monitored to ensure the absence of microbial contamination. All experiments were conducted under Cincinnati Children's Hospital Animal Care and Use Committee approved protocols.

### Fungi

The commonly used C. albicans wild-type laboratory strain SC5314 was a kind gift from Dr. Daniel H. Kaplan (University of Minnesota) (Igyarto et al., Immunity 35:260-272 (2011)). S. cerevisiae strain MYA797 was purchased from the ATCC.

### Viruses

Influenza A virus expressing OVA₂₅₇₋₂₆₄ (SIINFEKL) peptide (OVA-Influenza A) derived from the PR8 H1N1 strain was a kind gift from Drs. Thomas Moran (Ichan School of Medicine at Mount Sinai) and Paul Thomas (Saint Jude Children's Research Hospital).

### Experimental Replication, Randomization and Blinding

For each experiment, age and sex-matched groups of mice were randomly allocated to experimental groups. Each experiment was independently performed at least twice to ensure reproducibility. Histological scoring was performed by a board-certified veterinarian pathologist (T.A.) in a double blinded fashion. Sample sizes per group in each experiment reflect ≥80% power to detect a Cohen's effect size of 1.5 with an α error probability of 0.05 (G*Power 3.1).

### Antibiotic, Antimycotic and DSS Treatment

To eradicate commensal bacteria, filter-sterilized drinking water was supplemented with ampicillin (0.5 mg/mL, Sigma), gentamicin (0.5 mg/mL, Sigma), metronidazole (0.5 mg/mL Sigma), neomycin (0.5 mg/mL, Sigma), vancomycin (0.25 mg/mL, MP Biomedicals) and sucralose (4 mg/mL, Sigma) (Abt et al., Immunity 37:158-170 (2012); Elahi et al., Nature 504:158-162 (2013)). For depletion of intestinal C. albicans, the antibiotic cocktail was supplemented with fluconazole (0.5 mg/mL, Sigma) (Iliev et al., Science 336:1314-1317 (2012)). To induce intestinal injury, the drinking water of mice was supplemented with DSS (40,000 kDa, Alfa Aesar) for 6 days with or without the antibiotic cocktail, and then received untreated or antibiotic supplemented drinking water for the remainder of the experiment. Wild-type mice were administered 3% DSS. To account for the increased DSS susceptibility that occurs in the absence of dectin-1 or TLR4, or among gnotobiotic germ-free mice (Iliev et al., Science 336:1314-1317 (2012); Kitajima et al., Experimental animals 50:387-395 (2001); Rakoff-Nahoum et al., Cell 118:229-241 (2004)), 2% DSS was used for comparing the impacts of fungal colonization and/or commensal bacteria eradication in these animals.

### Fungal Colonization

Fungi were cultured the day prior in yeast extract-peptone-adenine-dextrose media at 30° C. (200 rpm). The following day, the culture was washed and suspended in sterile saline. Antibiotic treated mice were administered an oral lavage of 10⁶ fungal CFUs (in 30 μL phosphate-buffered saline) via P200 micropipette (Xin et al., Proc Natl Acad Sci USA 111:10672-10677 (2014)).

### Recoverable Bacterial or Fungal Burden

Tissues were sterilely collected, homogenized, and serial dilutions of each homogenate (into PBS) were spread onto brain heart infusion media (Sigma) agar plates. For isolating fungi, brain heart infusion media used for agar plates were supplemented with ampicillin (2.5 μg/mL, Sigma), gentamicin (2.5 μg/mL, Sigma), metronidazole (2.5 μg/mL Sigma), neomycin (2.5 μg/mL, Sigma), vancomycin (1.25 μg/mL, MP Biomedicals). Colony forming units were enumerated after incubation for 24 hours at 37° C.

DNA/RNA Isolation and qPCR

Bacterial DNA was isolated using the QIAamp DNA stool mini kit (Qiagen) according to the manufacturer's instructions. For isolating fungal DNA, individual fecal pellets were suspended in 50 mM Tris buffer (pH 7.5) supplemented with 1 mM EDTA, 0.2% β-mercaptoethanol and 1000 units/ml of lyticase (Sigma), incubated at 37° C. for 30 minutes to disrupt fungal cells as described (Iliev et al., Science 336:1314-1317 (2012)) prior to processing through the QIAamp DNA stool mini kit (Qiagen). Viral RNA was extracted with the QIAamp Viral RNA Mini Kit (QIAGEN) according to manufacturer's instructions. qPCR was performed on a 7500 Fast Real-Time PCR System (Applied Biosystems) using the following primers:

Bacterial 16S:
Forward
(SEQ ID NO: 1)
5′-ACTCCTACGGGAGGCAGCAGT-3′,
Reverse
(SEQ ID NO: 2)
5′-ATTACCGCGGCTGCTGGC-3′;
Fungal ITS1-2:
Forward
(SEQ ID NO: 3)
5′-CTTGGTCATTTAGAGGAAGTAA-3′,
Reverse
(SEQ ID NO: 4)
5′-GCTGCGTTCTTCATCGATGC-3′;
Eukaryotic 18S:
Forward
(SEQ ID NO: 5)
5′-ATTGGAGGGCAAGTCTGGTG-3′,
Reverse
(SEQ ID NO: 6)
5′-CCGATCCCTAGTCGGCATAG-3′;
MNV RdRp:
Forward
(SEQ ID NO: 7)
5′-CCAAAGTGGGATAGAAATGGTAGTC-3′,
Reverse
(SEQ ID NO: 8)
5′-TCACTCATCCTCATTCACAAGACT-3′.

### Intestinal Permeability

Fasting was initiated and maintained throughout the experiment starting 2 hours prior to intragastric gavage with 0.6 mg/g body weight of FITC-labeled dextran solution (from 100 mg/mL, FD4, Sigma). After 4 hours, blood from the retro-orbital sinus was collected in heparinized tubes and serum analysis for FITC concentration was performed with a fluorescence spectrophotometer (Synergy HTX, Biotek) at an excitation wavelength of 485 nm and emission wavelength of 528 nm. Standard curves were obtained by diluting FITC-dextran in saline.

### Influenza A Infection

OVA-Influenza A virus was grown and titered in Madin-Darby canine kidney epithelial cell monolayers, and stored at −70° C. For infection, individual virus aliquots were thawed, diluted in PBS, and administered intranasally (6×10⁴ PFUs in 30 μL) to mice anesthetized with xylazine and ketamine (Ichinohe et al., Proc Natl Acad Sci USA 108:5354-5359 (2011)).

### Microscopy

Hematoxylin and eosin (H&E) or alcian blue/periodic acid-Schiff (AB/PAS) staining of intestinal tissue was performed by the Pathology Core at Cincinnati Children's Hospital. Sections were imaged on a Nikon Eclipse 80i microscope. Slides were analyzed using ImageJ software. Villi width was measured where the base of the villi meets the crypt; at least 50 villi per mouse were measured for villi width. Paneth cell granules were counted in at least 30 crypts per mouse (Kernbauer et al., Nature 516:94-98 (2014)). Mean values were calculated for each mouse and used as individual data points. The histological scoring for DSS treated mice was graded on a severity scale of 1-5 for epithelial ulceration, inflammatory cell infiltration, and edema.

Isolation of Lymphocytes from Lung Parenchyma

Lungs were minced into small pieces with a razor and digested with collagenase D (1 mg/mL, Sigma), DNAse (0.1 mg/mL, Sigma) in DMEM (Gibco) supplemented with 10% (vol/vol) FBS, 1% (vol/vol) L-glutamine (Cellgro), 1% (vol/vol) penicillin-streptomycin (Cellgro) and 10 mM HEPES (Cellgro) for 60 minutes (37° C., 200 rpm), and then mashed through a 70 μm filter. Residual red blood cells were lysed with hypertonic solution (10 mM HKCO₃, 16 mM NH₄Cl, pH 7.3) prior to tetramer staining or cytokine stimulation.

### Tetramer Staining and Flow Cytometry

Single cell suspensions from the lung or mediastinal lymph node were incubated with brilliant violet 421-conjugated OVA₂₅₇₋₂₆₄:H2-K^{b} tetramer (60 minutes, 25° C.) prior to staining with the following fluorophore-conjugated antibodies purchased from eBioscience: FITC anti-mouse CD4 (clone GK1.5), APC anti-mouse CD8α (clone 53-6.7), PE-Cy5 anti-mouse CD11b (clone M1/70), PE-Cy5 anti-mouse CD11c (clone N418), PE-Cy5 anti-mouse F4/80 (clone BM8), PE-Cy5 anti-mouse B220 (clone RA3-6B2), eFluor 450 anti-mouse IFN-γ (clone XMG1.2). Samples were acquired on a BD FACSCanto and analyzed with FlowJo software (Treestar). OVA₂₅₇₋₂₆₄:H-2K^{b} specific CD8 T cells were gated on lymphocytes, single cells, B cell and myeloid (B220, CD11b, CD11c, F4/80) negative, CD8⁺CD4⁻ tetramer positive cells.

### Cytokine Production

Single cell suspensions from the lung were stimulated with 50 μM OVA₂₅₇₋₂₆₄ peptide in media supplemented with BD GolgiPlug (BD Biosciences) according to manufacturer's instructions for 4-5 hours at 37° C.

Rectal Inoculation with Fungal Cell Wall Moieties

Mice were intrarectally administered the indicated dosage of mannan (Sigma), curdlan (Wako) or zymosan (Sigma) suspended in 50 μl saline after anesthetization with xylazine and ketamine beginning one day prior to DSS or influenza A challenge, and re-administered every other day thereafter.

### RAW-Blue Stimulation and Quantification of NF-κB Activity

RAW-Blue cells (InvivoGen) cells are derived from the murine RAW 264.7 macrophages with chromosomal integration of a secreted embryonic alkaline phosphatase reporter construct induced by NF-κB. 5×10⁴ RAW-Blue cells were seeded 24 hours prior to addition of the indicated blocking antibody. One hour thereafter, mannan (Sigma) was added at a final concentration of 500 μg/mL. Supernatants were collected after 24 hours to quantify NF-κB activity by colorimetric assay using QUANTI-Blue reagent (InvivoGen).

### Quantification and Statistical Analysis

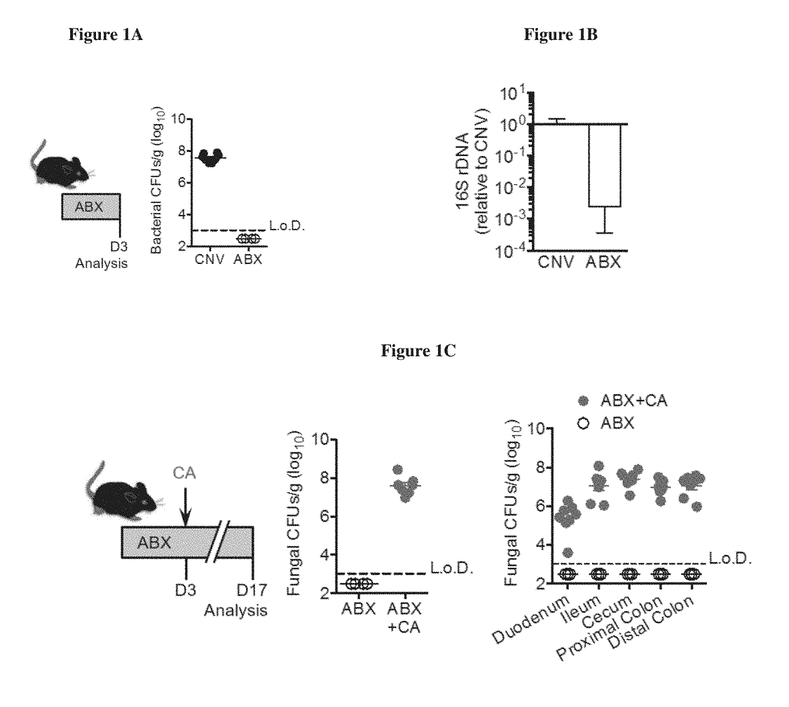

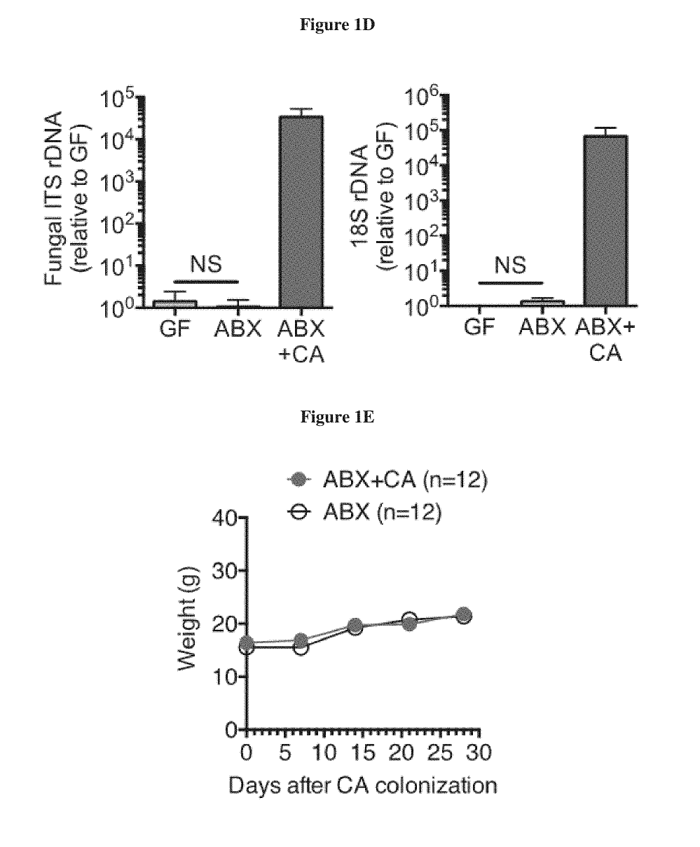

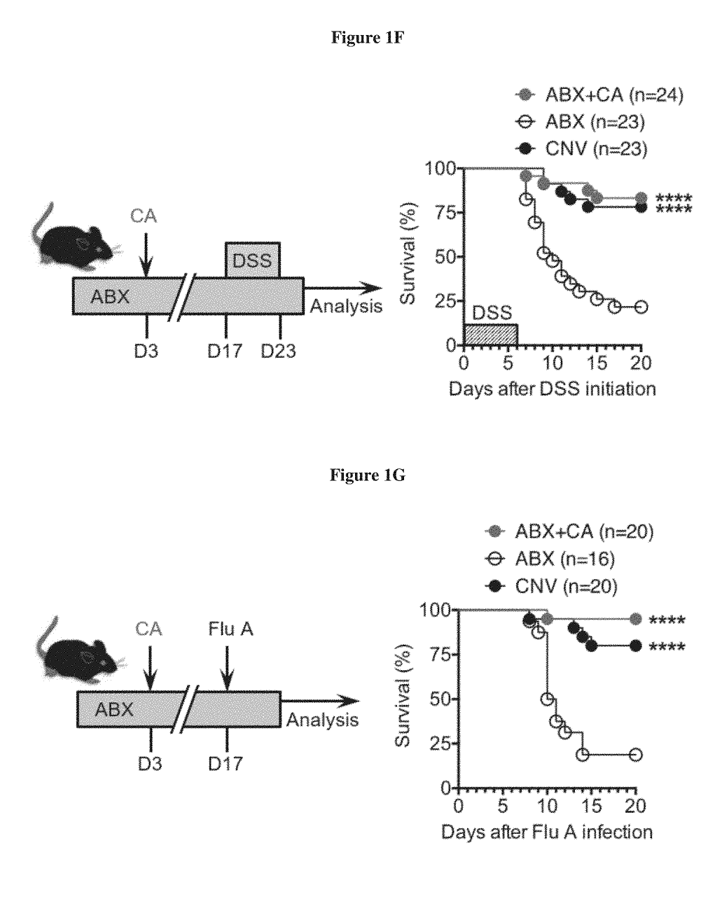

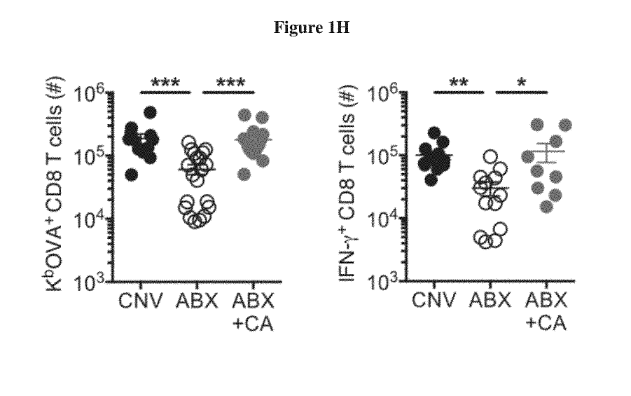

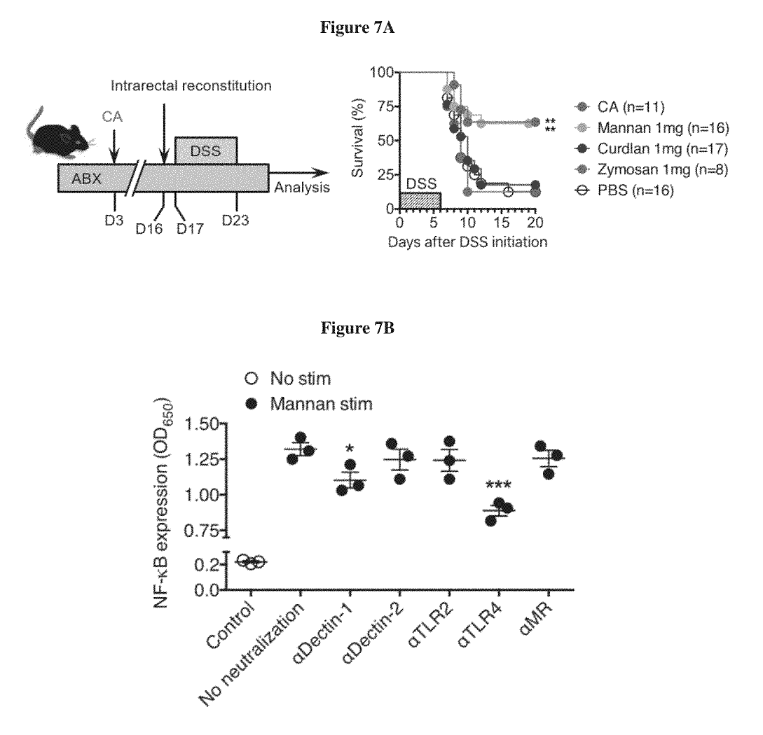

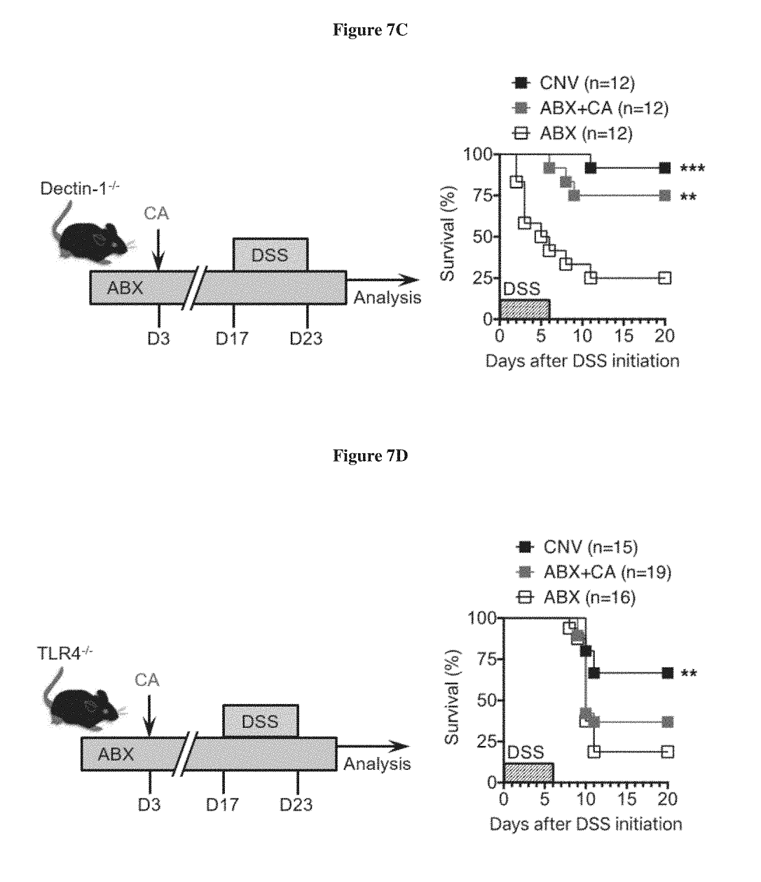

The number (n) of individual animals used per group are described in each individual figure panel, or shown by individual data points that represents the results from an individual animal (FIGS. 1A-1H, 2A-2E, FIGS. 3A-3D, FIGS. 4A-4B, FIGS. 5A-5E, and FIGS. 8A-8B) or individual well for cell stimulation assays (FIGS. 7A-7D). The number of replicate experiments is described in each figure legend. All statistics and data distribution analysis were performed with Prism (GraphPad). The unpaired two-tailed Student's t-test with Welch's correction was used to compare differences between two groups (Ruxton, Behavioral Ecology 17:688-690 (2006)). The non-parametric Kruskal-Wallis test with Dunn's correction, one-way ANOVA with Holm-Sidak's correction or Mann-Whitney U test was used to evaluate experiments containing more than two groups depending on the distribution pattern of the data. Survival curves were analyzed by the Log-rank (Mantel-Cox) test. The upper threshold for statistical significance for all experiments was set at P<0.05.

### Results

C. albicans Intestinal Mono-Colonization Overrides the Protective Necessity of Commensal Bacteria

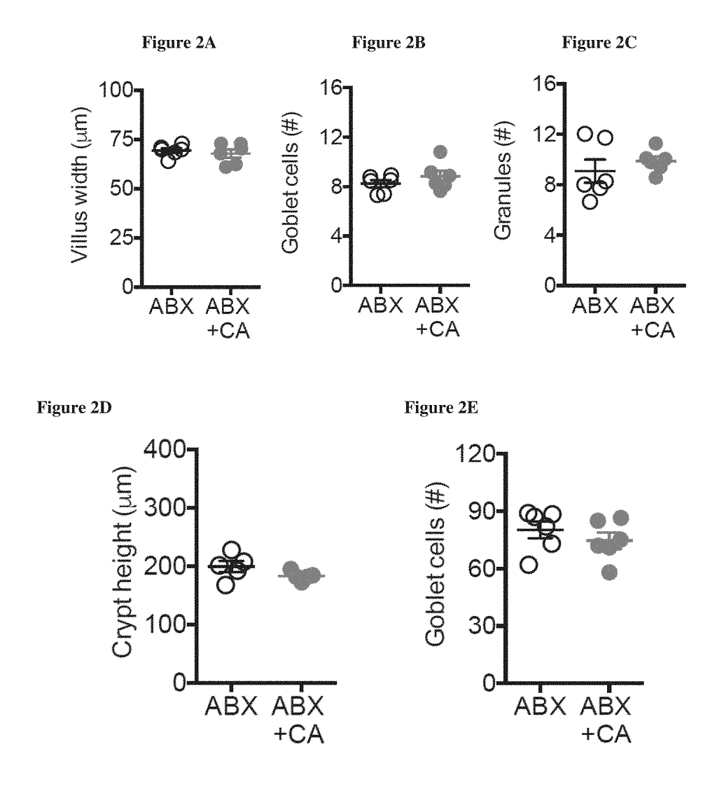

Fungi are naturally impervious to antibiotics that eradicate commensal bacteria, and therefore are poised to bloom during antibiotic treatment (Erb Downward et al., Sci Rep 3:2191 (2013); Fan et al., Nat Med 21:808-814 (2015); Mason et al., Infect Immun 80:3371-3380 (2012)). This ability of fungi to rapidly accumulate after antibiotic induced eradication of intestinal bacteria was exploited to investigate whether commensal fungi—in isolation—can functionally recapitulate the protective benefits of enteric bacteria. Supplementing the drinking water with a previously described cocktail of broad-spectrum antibiotics efficiently eliminates recoverable anaerobic and aerobic bacteria from the feces of mice housed in a specific pathogen free facility (FIG. 1A) (Abt et al., Immunity 37:158-170 (2012); Jiang et al., Mucosal Immunol 8:886-895 (2015)). 16S rDNA copies were also sharply reduced (>400-fold) in the feces of antibiotic treated compared with control mice (FIG. 1B), in agreement with prior studies demonstrating commercial rodent chow subsequently becomes the major contributor and source of residual bacterial 16S rDNA in mice receiving this antibiotic cocktail (Hill et al., Mucosal immunology 3:148 (2010)). In turn, C. albicans efficiently establishes intestinal colonization among mice sustained on this broad-spectrum antibiotic cocktail (FIG. 1C). Interestingly, despite high-density intestinal C. albicans colonization, mice gained weight at a comparable tempo compared with antibiotic treated controls not administered C. albicans (FIG. 1D). Fungal intestinal colonization also occurs without disrupting the architecture and morphology of local tissues. Crypt height, goblet cell density and paneth cell granularity were each histologically unchanged throughout the small and large intestine among C. albicans colonized mice (FIGS. 2A-2E). Together, these results show C. albicans can replace intestinal bacteria in a commensal fashion without overt harmful consequences.

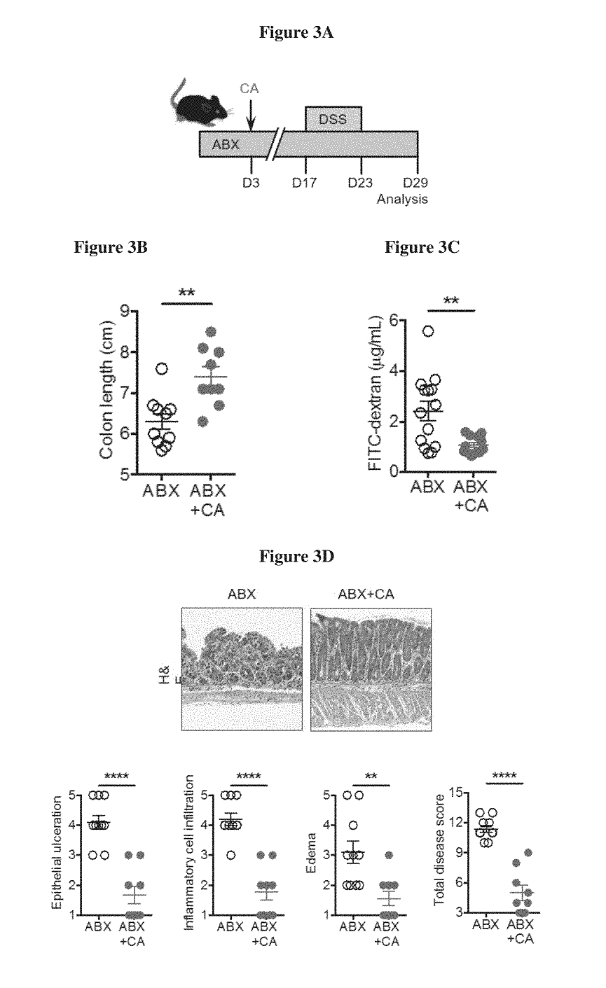

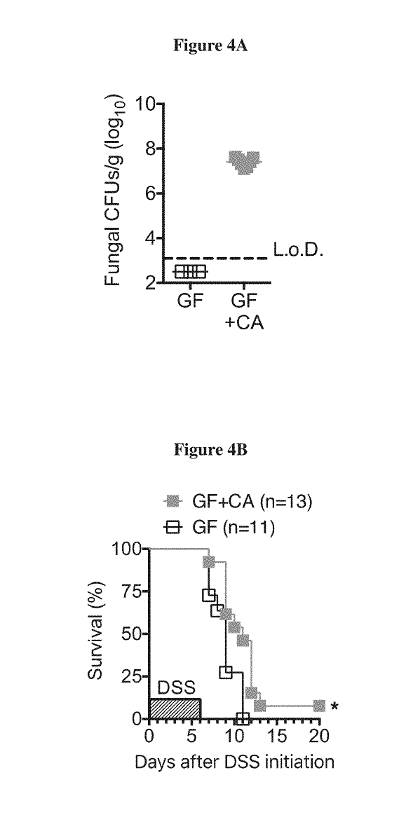

Mice housed in a specific pathogen free facility are devoid of endogenous commensal fungi and noroviruses since the levels of these microbes measured in the feces by nucleic acid qPCR were comparable to the background observed for germ-free mice (FIG. 1E) It was reasoned that any in vivo shifts in disease susceptibility will reflect the biological properties of mono-colonization with this fungal species. Remarkably, C. albicans mono-colonization efficiently overturned mortality induced by DSS among antibiotic treated mice, with overall survival and average time-to-death rebounding to levels comparable to commensal bacteria replete conventional mice (FIG. 1F). Commensal C. albicans also protects against DSS induced colonic shortening, intestinal permeability and inflammation compared with antibiotic treated control mice (FIGS. 3A-3B). Importantly, this protection cannot be explained by potential bacteria that persist despite antibiotic treatment since C. albicans mono-association also significantly improved survival of germ-free mice, which are highly susceptible to DSS induced intestinal injury (Kitajima et al., Experimental animals 50:387-395 (2001)) (FIGS. 4A-4B). Thus, enteric mono-colonization with a single fungal species can override the protective necessity of commensal bacteria in averting local tissue injury.

The immune modulatory properties of commensal fungi in extra-intestinal tissues were evaluated, given the active calibration of systemic immune cell responsiveness previously shown for intestinal bacteria (Abt et al., Immunity 37:158-170 (2012); Clarke et al., Nat Med 16:228-231 (2010); Ichinohe et al., Proc Natl Acad Sci USA 108:5354-5359 (2011)). For these studies, influenza A virus with restricted tropism to respiratory tissues (Chaturvedi et al., J Clin Invest 125:1713-1725 (2015)), and natural resistance to the antibiotics used to facilitate C. albicans colonization (Abt et al., Immunity 37:158-170 (2012); Ichinohe et al., Proc Natl Acad Sci USA 108:5354-5359 (2011)), was used as it makes it an ideal pathogen to probe the extra-intestinal impacts of commensal fungi. It was found that C. albicans mono-colonization efficiently overturned the fatal susceptibility to influenza A virus infection amongst commensal bacteria depleted mice (FIG. 1G). Commensal fungi also reversed the blunted accumulation and IFN-γ production of viral-specific CD8 T cells for mice treated with antibiotics, to levels comparable to commensal bacteria replete conventional mice housed under specific pathogen free conditions (FIG. 1H). Collectively, these findings highlight C. albicans mono-colonization can replace the systemic benefits of commensal enteric bacteria and C. albicans intestinal mono-colonization overrides the protective necessity of commensal bacteria.

S. cerevisiae Intestinal Mono-Colonization Overturns Disease Susceptibility Induced by Commensal Bacteria Depletion

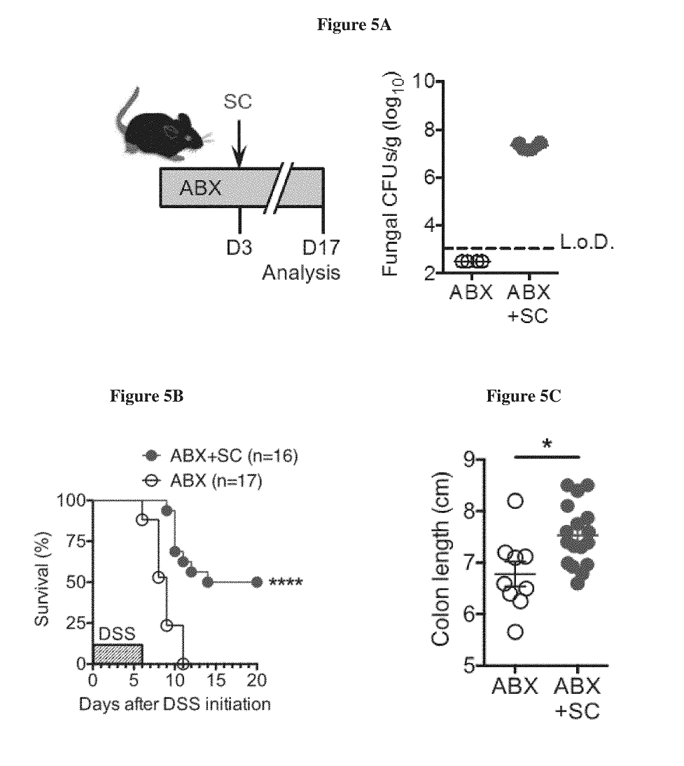

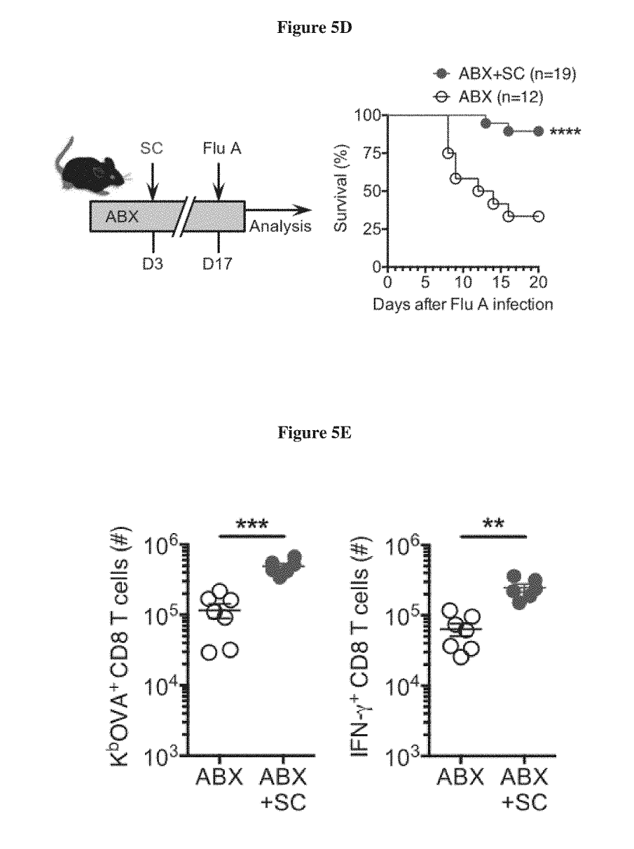

S. cerevisiae, another yeast naturally found in the mammalian gut (Hoffmann et al., PLoS One 8:e66019 (2013); Sokol et al., Gut 66:1039-1048 (2017)), was used to evaluate whether these protective benefits are shared by other species of commensal fungi. Similar to C. albicans, mice treated with the same cocktail of broad-spectrum antibiotics and then inoculated with S. cerevisiae consistently have high-density of this yeast in their feces (FIG. 5A). In turn, the uniform mortality and colonic shortening induced by DSS among commensal bacteria depleted mice was dramatically improved in mice with S. cerevisiae mono-colonization (FIGS. 5B and 5C). Commensal S. cerevisiae also reduced susceptibility to intranasal influenza A virus infection among antibiotic treated mice (FIG. 5D), which coincided with rebounded accumulation and IFN-γ production by protective viral-specific CD8 T cells (FIG. 5E). These near identical benefits conferred by S. cerevisiae and C. albicans suggest universal protective properties shared across fungal species capable of mammalian host colonization.

### Protective Benefits of Enteric Commensal Fungi Require Persistent Colonization

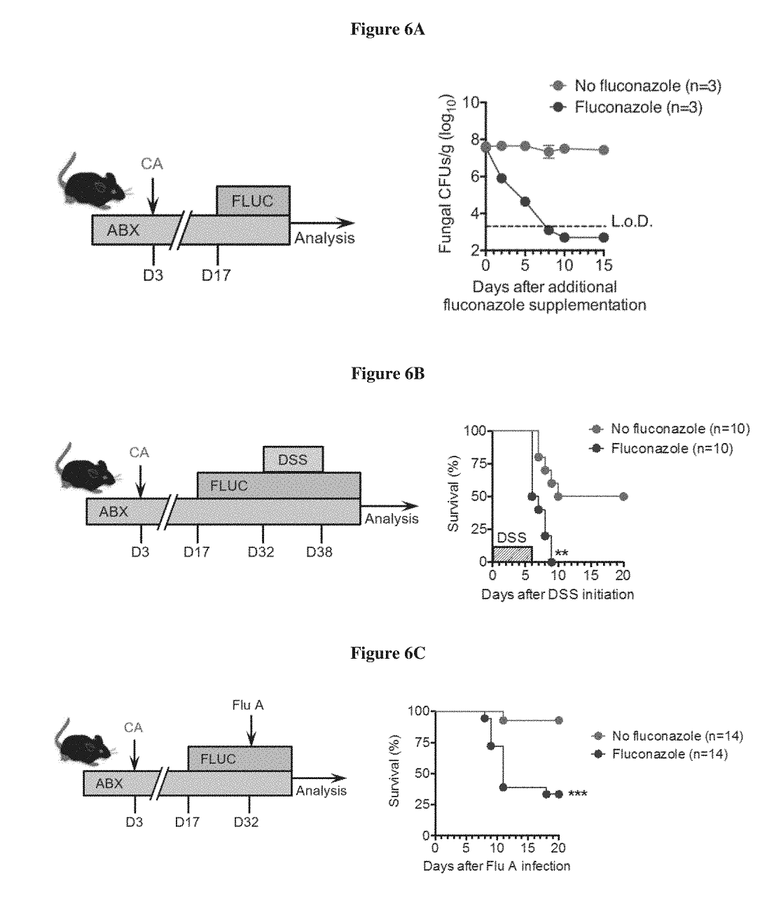

To investigate the durability of protection conferred by commensal fungi, the impacts of fungal eradication with the antimycotic agent, fluconazole, on protection against DSS colitis and respiratory influenza A virus infection were evaluated. It was found that recoverable fungi in the feces of C. albicans mono-colonized mice rapidly declined to undetectable levels 10 days after adding fluconazole to antibiotic-supplemented drinking water that is consistent with recent studies (FIG. 6A) (Underhill and Lliev, Nature Reviews Immunology 14:405-416 (2014); Wheeler et al., Cell Host Microbe 19:865-873 (2016)). Such, fungal eradication efficiently abolished the improved survival and delayed time-to-death after DSS challenge conferred by C. albicans mono-colonization (FIG. 6B). Protection against influenza A was similarly overturned following depletion of commensal fungi (FIG. 6C). This necessity for persistent fungal colonization in protection against DSS colitis and influenza A virus infection is consistent with susceptibility to chemical colitis and allergic airway disease unleashed after fluconazole treatment of mice with a diverse repertoire of commensal microbe (Wheeler et al., Cell Host Microbe 19:865-873 (2016)). However, this requirement for tonic presence of commensal fungi in systemic immune modulation contrast with the concept of “trained immunity” recently shown to be primed by invasive fungi (Cheng et al., Science 345:1250684 (2014)), which may reflect contextual differences in sensing fungi as intestinal microbes as opposed to pathogens in sterile tissues after parental injection.

### Mannans Mediate the Protective Benefits Conferred by Commensal Intestinal Fungi

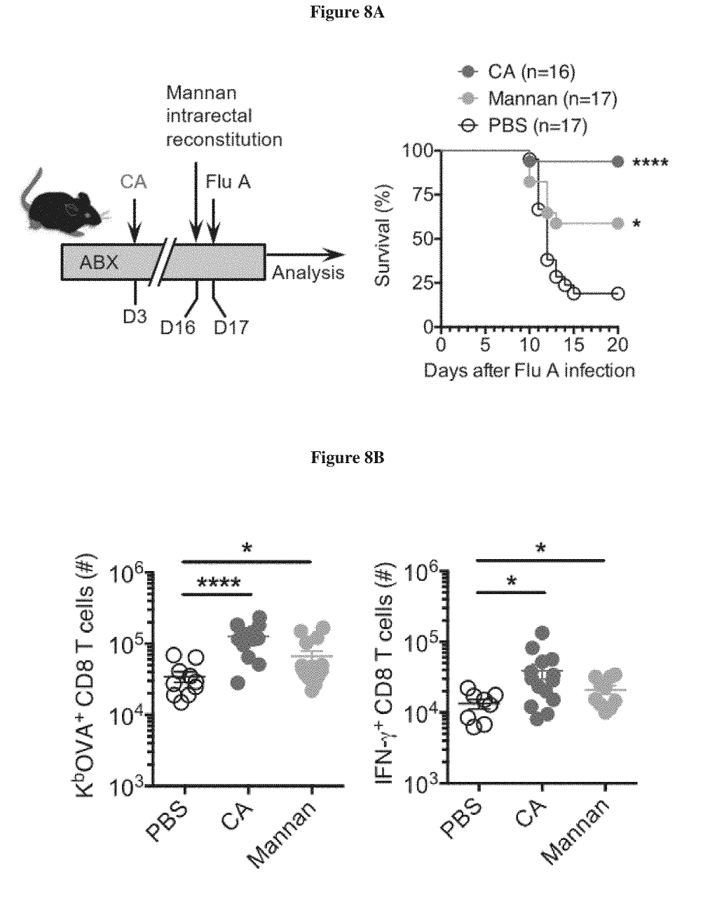

The function aspect of the commensal fungi was evaluated, by testing if mannans, a major cell wall component shared by all fungi, may be responsible. It was shown that this fungal moiety could functionally replace the protective benefits of intact fungi in the absence of commensal bacteria. Intrarectal administration was employed to mimic the high-density commensal fungi colonization observed in the lower intestinal tract. Susceptibility to DSS and influenza A virus infection among antibiotic treated mice were each mitigated by mannan reconstitution, with survival and accumulation of protective immune cells restored to levels comparable to C. albicans colonized controls (FIG. 7A and FIG. 8). By contrast, intrarectal inoculation with other fungal cell wall components such as curdlan or zymosan failed to improve mortality among antibiotic treated mice (FIG. 7A), showing that the protective benefits of commensal fungi are recapitulated by mannans, a highly conserved fungal cell wall constituent.

Fungal derived mannans can stimulate host cells through a variety of microbial pattern recognition receptors. Therefore, the necessity of each host receptor was investigated. Through recognition of a purified, biologically active mannan preparation, it was assessed how neutralizing each molecule impacts mannan stimulation in vitro. For these studies, the RAW-Blue 264.7 macrophage NF-κB reporter cell line was used, which are responsive to stimulation through a wide variety classical microbial pattern recognition receptors including toll-like receptors and c-type lectins (i.e., dectin-1, dectin-2, mannose receptor) (Bi et al., Journal of Biological Chemistry 285:25969-25977 (2010); Ying et al., The Journal of Immunology 194:1239-1251 (2015)). Interestingly, neutralization of dectin-1 and TLR4 each significantly reduced mannan induced NF-κB. By contrast, mannan stimulated cell activation was not significantly impacted by antibody blockade against TLR2, dectin-2 or the mannose receptor (FIG. 7B).

The in vivo necessity of dectin-1 and TLR4 in mediating the protective benefits of C. albicans was investigated by evaluating the impact of fungal colonization on susceptibility to DSS induced intestinal injury among mice with targeted defects in these molecules. Similar to isogenic WT mice on the C57BL/6 background, susceptibility to DSS was sharply increased after antibiotic induced eradication of commensal bacteria in both dectin-1-deficient and TLR4-deficient mice (FIG. 7C, 7D). Interestingly, C. albicans mono-colonization efficiently overturned DSS induced mortality among dectin-1 deficient mice (FIG. 7C). By contrast, the protective benefits of fungal colonization against DSS were sharply reduced among TLR4 deficient mice, with only marginally improved survival amongst C. albicans colonized mice compared with antibiotic treated controls (P=0.90) (FIG. 7D). Thus, TLR4 plays essential non-redundant roles for conferring the protective benefits of commensal C. albicans that bypass the protective necessity of enteric bacteria through fungal specific mannans.

### Discussions

Tonic stimulation by commensal bacteria is increasingly recognized to improve many aspects of host health (Abt et al., Immunity 37:158-170 (2012); Chung and Kasper, Current opinion in immunology 22:455-460 (2010); Hooper et al., Science 336:1268-1273 (2012); Ichinohe et al., Proc Natl Acad Sci USA 108:5354-5359 (2011); Rakoff-Nahoum et al., Cell 118:229-241 (2004); Round and Mazmanian, Nat Rev Immunol 9:313-323 (2009)). This examples shows that, unexpectedly, susceptibility to intestinal injury and extra-intestinal infection caused by absent bacteria is overturned with fungal colonization. Antifungal administration eliminates the beneficial impacts of commensal fungi, in agreement with the necessity for endogenous enteric fungi to protect against intestinal injury or airway inflammation in the presence of commensal bacteria (Wheeler et al., Cell Host Microbe 19:865-873 (2016)). These benefits of commensal fungi, when evaluated in the absence of intestinal bacteria, are in sharp contrast to their deleterious roles in exacerbating intestinal injury in dectin-1 deficient mice (Iliev et al., Science 336:1314-1317 (2012)). This discrepancy likely reflects additional stimulation by commensal bacteria or differences in commensal bacteria composition among dectin-1 deficient mice (Tang et al., Cell Host Microbe 18:183-197 (2015)), or discordant features of the fungal “mycobiome” across institutions (Hiev et al., Science 336:1314-1317 (2012)). By exploiting the absence of detectable endogenous fungi, this Example demonstrated that individual fungal species, in isolation, can take the place of commensal bacteria in positively calibrating local and systemic immunity.

It was also identified herein that the protective benefits of commensal fungi are mediated by mannan, a highly conserved structural component of fungal cell walls. These results parallel the biological properties of commensal bacteria conferred by their principal molecular constituents. For example, lipotechoic acid or LPS administration, in lieu of live commensal bacteria, can each avert DSS-induced mortality (Rakoff-Nahoum et al., Cell 118:229-241 (2004)). Likewise, peptidoglycan, LPS, CpG or poly(I:C) reconstitution augments systemic antimicrobial immunity among antibiotic treated mice (Abt et al., Immunity 37:158-170 (2012); Ichinohe et al., Proc Natl Acad Sci USA 108:5354-5359 (2011)). These benefits of commensal bacteria conferred by their individual structural components, require host microbial pattern recognition receptors as LPS-mediated protection against DSS colitis is abolished in absence of TLR4 (Rakoff-Nahoum et al., Cell 118:229-241 (2004)). Interestingly however, host recognition of mannans has been demonstrated to occur with considerably more functional redundancy spanning multiple pattern recognition receptors including TLR2, TLR4, dectin-1, dectin-2, DC-SIGN, mincle and the mannose receptor (Hardison and Brown, Nat Immunol 13:817-822 (2012); Jouault et al., J Infect Dis 188:165-172 (2003); Netea et al., Nat Rev Microbiol 6:67-78 (2008); Nigou et al., J Immunol 180:6696-6702 (2008); Saijo et al., Immunity 32:681-691 (2010)). While these conclusions have been shown using complementary models of in vitro stimulation with purified mannans or parenteral infection with invasive fungal pathogens, the data presented herein show an essential role for TLR4 in sensing commensal fungi in vivo.

In sum, this study shows the protective benefits of commensal microbes are not limited to bacteria and viruses, but also shared by enteric fungi.

### Example 2: Commensal Candida albicans Positively Calibrate Systemic Th17 Immunological Responses

Colonization with pathobiont microbes is a classical risk factor for invasive infection. However, the relative paucity of systemic infection despite ubiquitous pathobiont commensalism suggests colonization may also elicit protective host immunity. Here it is shown that C. albicans intestinal colonization in mice and humans drives systemic expansion of fungal-specific Th17 CD4+ T cells, and IL-17 responsiveness by circulating neutrophils, which synergistically protect against C. albicans invasive infection. Persistent C. albicans colonization is required, since protection is overturned by eradicating fungal colonization. Commensal C. albicans conferred protection extends to invasive infection by the extracellular bacterial pathogen, Staphylococcus aureus. However, positively calibrating systemic Th17 responses is not uniformly beneficial, as commensal C. albicans does not protect against intracellular influenza A virus infection, and exacerbates allergic airway inflammation susceptibility. Thus, systemic Th17 inflammation driven by CD4+ T cells responsive to commensal C. albicans tonic stimulation improves host defense against extracellular pathogens, but with harmful immunological consequences.

### Material and Methods

### Mice

C57BL/6 mice were purchased from Charles River laboratories, and housed under specific pathogen-free conditions, and used between 6-8 weeks of age. All experiments were performed using sex- and age-matched controls under Cincinnati Children's Hospital Research Foundation IACUC approved protocols.

### Microbes

Wildtype C. albicans (strain SC5314) and the isogenic recombinant virulent strain expressing GFP plus 2W1S55-68 peptide was provided by Dr. Daniel Kaplan (University of Pittsburgh) (Igyártó et al., Immunity 35:260-272 (2011)). Methicillin resistant S. aureus (strain USA300) was provided by Dr. Matthew Flick (Cincinnati Children's Hospital). Mouse adapted H1N1 influenza A virus (strain PR8) was provided by Monica Malone McNeal (Cincinnati Children's Hospital).

### Antibiotic and Antimycotic Agents

To establish C. albicans intestinal colonization, the drinking water of mice was supplemented with ampicillin (1 mg/ml) two days prior to oral C. albicans inoculation. Thereafter mice were maintained on ampicillin supplemented drinking water throughout the experiment. To eradicate commensal fungi, ampicillin treated drinking water was further supplemented with fluconazole (0.5 mg/ml).

### Microbial Propagation and Infection

For oral inoculation, each C. albicans strain (wildtype or recombinant) was cultured in YPAD media at 30° C. overnight with shaking (200 rpm). The following day, 30 μl of the overnight culture was administered dropwise into the mouths of mice. For intravenous infection, each C. albicans strain (wildtype or recombinant) was grown in YPAD media at 30° C. with shaking (200 rpm), back-diluted to early log phase growth (OD600 ˜0.1), washed and diluted in sterile saline to the desired inoculum (5×10⁴ CFUs/200 μl) and injected into mice via the lateral tail vein. For infection, S. aureus was grown in BHI media at 37° C. with shaking (200 rpm), back-diluted to early log phase growth (OD600 ˜0.1), washed and diluted in sterile saline to the desired inoculum (10⁸ CFUs/200 μl or 3×10⁷ CFUs/200 μl). For influenza A virus infection, frozen aliquots of PR8 virus were individually thawed, diluted in saline to 2×10⁵ PFU/30 μl, and intranasally administered to anesthetized (ketamine/xylazine) mice (Jiang et al., Cell host & microbe 22:809-816 (2017)). Mice were checked daily and assigned the following clinical disease score (1 healthy; 2 limited ruffled fur; 3 ruffled fur throughout; 4 mild lethargy; 5 limited movement; 6 moribund or uncontrolled spastic movements; 7 deceased) as described (Turner et al., PLoS pathogens 13:e1006684 (2017)). For enumerating the number of recoverable C. albicans and S. aureus colony forming units, individual fetal pellets or each tissue from mice was sterilely dissected, weighed, and homogenized in sterile saline. Serial dilutions on the organ homogenate were spread onto BHI media plates (S. aureus) or BHI media plates supplemented with antibiotics (C. albicans) (ampicillin [2.5 μg/mL), gentamicin (2.5 μg/mL), metronidazole (2.5 μg/mL), neomycin (2.5 μg/mL), and vancomycin (1.25 μg/mL), and the number of individual colonies enumerated after incubation at 37° C., for 24 hours.

### Cell Staining, Stimulation and Flow Cytometry

Fluorophore- or biotin-conjugated antibodies used mouse cell analysis are as follows: anti-CD4 (GK1.5, eBioscience), anti-CD8a (53-6.7, eBioscience), anti-CD11b (M1/70, Biolegend), anti-CD11c (N418, Biolegend), anti-F4/80 (BM8, eBioscience), anti-B220 (RA3-6B2, Biolegend), anti-CD44 (IM7, Biolegend), anti-CD45 (30F11, invitrogen), anti-Ly6G (1A8, BD bioscience), anti-Ly6C (HK1.4, Biolegend), anti-Gr1 (RB6-8C5, eBioscience), anti-IL-17RC (polyclonal IgG, R&D Systems), anti-FOXP3 (FJK-16S, eBioscience), anti-RORγt (Q31-378, BD bioscience), anti-T-bet (4B10, Biolegend), anti-IFN-γ (XMG1.2, eBioscience), anti-IL17A (TC 11-18H10.1, Biolegend), anti-IL17F (9D3.1C8, Biolegend). Fluorophore-conjugated antibodies were used for human cell analysis are as follows: anti-CD4 (OKT4, eBioscience), anti-CD15 (H198, invitrogen), anti-CD16 (3G8, Biolegend), anti-IL-17A (eBio64DEC17, eBioscience), anti-IL17F (O33-782, BD Bioscience), anti-IL17RC (309822, R&D Systems) with staining in the presence of Human Fc block (BD Bioscience). For detecting cytokine production by individual cells, 106 cells from the spleen and peripheral lymph nodes were stimulated with heat-killed (65° C., for 90 minutes) C. albicans (10⁶ CFU equivalents) in 200 μl complete DMEM medium at 37° C., and 5% CO2 for 24 hours, and with GolgiPlug (BD Biosciences) supplementation to the media for the last 6 hours, and intracellular staining was performed after cell permeabilization (BD PharMingen) according to the manufacturers' instructions. Single cell suspensions from spleen and peripheral lymph nodes were stained with APC conjugated I-Ab:2W1S55-68 tetramer at room temperature for 60 minutes, and enriched using anti-APC antibody conjugated magnetic beads (Militenyi Biotec) as described (Moon et al., Immunity 27:203-213 (2007)). Samples were acquired using an FACSCanto (BD) cytometer and analyzed using FlowJo software (Tree Star).

### In Vivo Cell Depletion or Cytokine Neutralization

The following antibodies were administered to mice for in vivo cell depletion or cytokine neutralization: anti-CD4 (GK1.5, BioXcell); anti-IL-17A (17F3, BioXcell), anti-IL17F (MM17F8F5, BioXcell), anti-Gr1 (RB6-8C5, BioXcell), anti-Ly6G (1A8, BioXcell), or each respective isotype antibody control (mouse IgG1, rat IgG2b, BioXcell) by intraperitoneal injection (0.6 μg each antibody per mouse) beginning 1 day prior to C. albicans intravenous infection or 7 days prior to blood collection. Thereafter, 0.3 μg of the same antibody was administered every two days until the end of the experiment.

### Imaging

BHI agar plates containing fungal colonies were imaged by the In Vivo Imaging System (Perkin Elmer) using the GFP and Cy5.5 filter sets. Fluorescence intensity was quantified by region-of-interest calibration using the Cy5.5-filter set image as background and showing the ratio properties as pseudo-color images (rainbow pseudo-color shows ratio high to low as red to blue), and analyzed using Nikon Elements software.

### Reactive Oxygen Production

Peripheral blood was collected via retro-orbital bleeding from mice. To detect production of reactive oxygen species, RBC lysed peripheral blood cells were seeded into 96-well round-bottom plates (2×10⁵/well) in the presence of 0.45 μM dihydrorhodamine 123 (Sigma) for 15 minutes, then stimulated with 100 μg/mL C. albicans extract (Greer Laboratories) for 60 minutes.

### Airway Reactivity and Inflammation

Mice were anesthetized with ketamine/xylazine and hung by their front incisors on an angled stand. 100 μg house dust mite extract (Greer Laboratories) in 40 μL sterile saline was administered intratracheally. 10 days later, mice were challenged with the same amount of house dust mite extract. 48 hours after the second intratracheal challenge, airway resistance in response to inhaled methacholine (100 mg/mL) was measured using FlexiVent (SCIREQ). Saline perfused lungs were minced and digested with collagenase D (1 mg/mL) and DNAse (0.1 mg/mL) in DMEM media supplemented with 10% fetal bovine serum, 1% L-glutamine, 10 mM HEPES, 1% penicillin-streptomycin (Cellgro) for 30 minutes at 37° C. with gentle shaking (200 rpm). The tissue digest was then passed through a 70 μm filter to obtain single cell suspensions. For intracellular cytokine staining, single cell preparations from the lung were stimulated with phorbol 12-myristate 13-acetate (PMA) and ionomycin (eBioscience Cell Stimulation Cocktail) for 4.5 hours at 37° C., and 5% CO2 in the presence of BD GolgiPlug (BD Biosciences).

### Human Sample Collection and Processing

Informed consent to use discard anticoagulated blood collected for routine clinical care was obtained under Cincinnati Children's Hospital Institutional Review Board (IRB) approved protocols. Inclusion criteria were patients scheduled for routine early morning blood collection so that analysis could be performed on excess sample after clinical processing, and those willing and able to provide a stool specimen. Exclusion criteria were patients with lymphopenia or neutropenia (absolute lymphocyte and neutrophil count each >500×10³/μl), recent (past 30 days) exposure to antifungal compounds, or being critically ill that precluded being able to obtain oral and written consent. Anticoagulated blood was RBC lysed, and peripheral blood mononuclear cells seeded into 96-well round-bottom plates (2×10⁵/well). Thereafter, triplicate wells were stimulated with heat-killed C. albicans (10⁶ CFU equivalents) and anti-human CD28 (5 μg/mL, BD Bioscience) or anti-human CD28 for 24 hours at 37° C., and with supplementation of the media with GolgiPlug (BD Biosciences) for the last 6 hours.

### Fecal DNA Extraction and Shotgun Metagenome Sequencing

DNA extraction was performed by mixing 0.1 gram stool with 0.35 mL Epicentre Masterpure Yeast DNA Purification lysis buffer to which was added 3500 U of Epicenter Ready Lyse solution. Two 5-mm stainless steel beads were added and the samples were vortexed for 1 hour at room temperature. The samples were frozen at −80° C. briefly then thawed and the supernatant was transferred to a new tube, to which 0.15 mL MPC Protein Precipitation Reagent was as added. After centrifugation (13,000×g) for 10 minutes the supernatant was added to new tube to which 0.2 mL 100% ethanol was added. The samples were incubated at −20° C., for 30 min. DNA was then purified on Invitrogen PureLink DNA columns following the manufacturer's instructions. DNA concentration was determined using Qubit analyzer, and diluted to 200 ng/mL. Nextera XT adapters following manufacturer's instructions, and sequencing was performed on an Illumina NextSeq500 machine using 150-bp DNA paired end reads to a depth of approximately 2.5 G base pairs per sample. Raw sequence data was demultiplexed and converted to fasta format and subjected to downstream analysis.

### Taxonomic Assignment of DNA Reads

Paired-end sequencing reads from each sample were aligned with Kraken (Wood and Salzberg, Genome biology 15:R46 (2014)) against an extensive microbial genome database comprised of all microbial and archaeal genomes deposited in RefSeq as of June 2017. The database was supplemented with greater than 4,000 additional finished published genomes derived from fungi, bacteria, and viruses not represented in RefSeq. Species count data was then converted into percent abundance per sample and exported in tabular format.

### Results

C. albicans Intestinal Colonization Protects Against Systemic C. albicans Infection

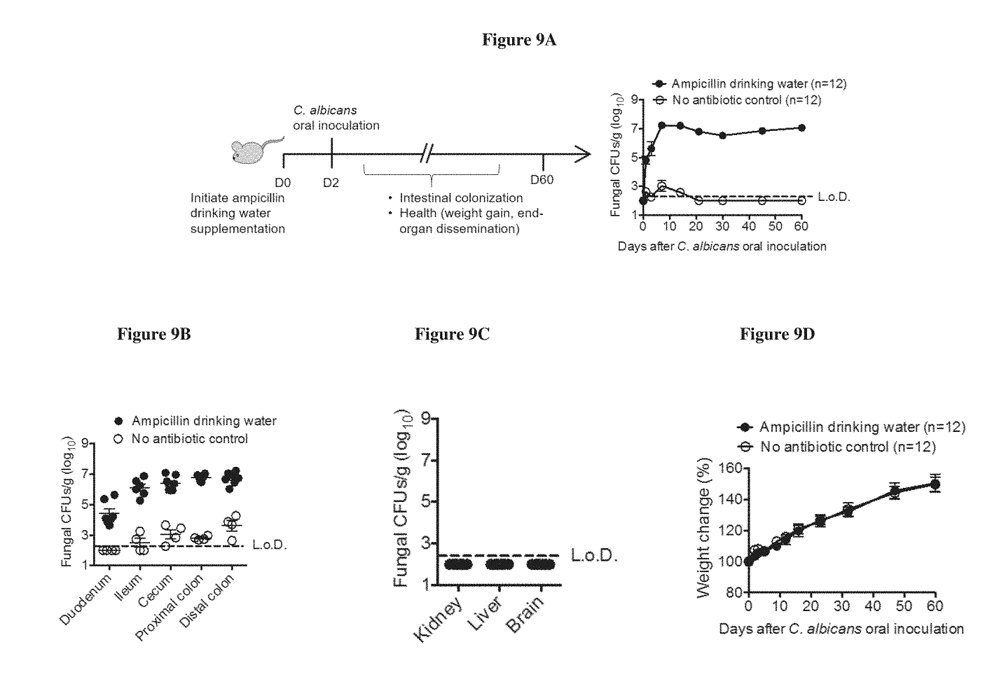

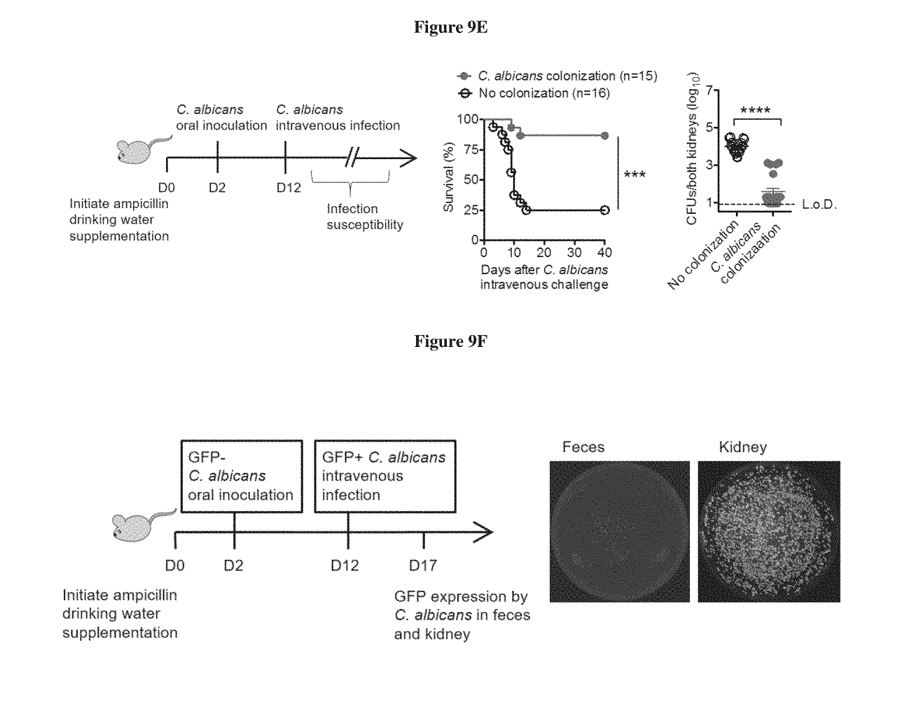

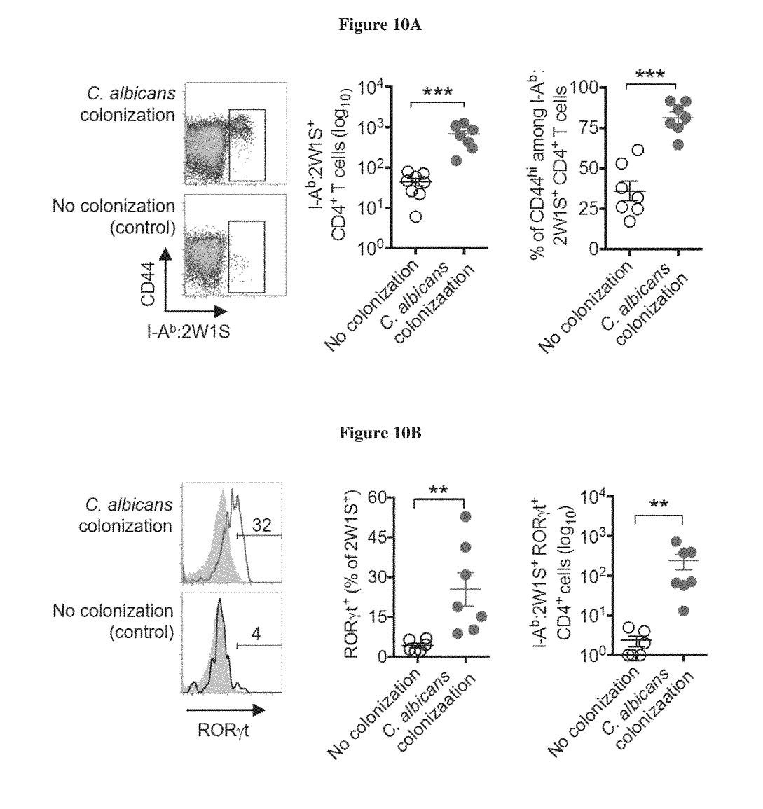

C. albicans is a common commensal of the human intestine. However, this fungal species is only rarely found in the feces of laboratory mice (Iliev et al., Science 336:1314-1317 (2012); Skalski et al., PLoS pathogens 14:e1007260 (2018); Wheeler et al., Cell host & microbe 19:865-873 (2016)), and a complete absence of C. albicans and other endogenous commensal fungi was shown for mice housed in a specific pathogen free facility (Jiang et al., Cell host & microbe 22:809-816 (2017)). It was reasoned the lack of commensal C. albicans in the colony could be exploited to investigate how long-term intestinal colonization with this pathobiont impacts host susceptibility to systemic C. albicans invasive infection. In turn, using recombinant C. albicans engineered to express defined model antigens (Igyártó et al., Immunity 35:260-272 (2011)) for establishing intestinal colonization would create an instructive model whereby adaptive immune components responsible for these shifts in invasive infection susceptibility could be precisely identified. To facilitate intestinal colonization, the drinking water of mice was supplemented with ampicillin (Samonis et al., Antimicrobial agents and chemotherapy 38:602-603 (1994)). It was found that a single oral inoculation of C. albicans administered to mice maintained on ampicillin supplemented drinking water results in sustained (>60 days) C. albicans intestinal colonization (FIG. 9A and FIG. 9B). C. albicans recovery in the feces was consistently achieved within 24 hours after oral inoculation, and this level further increased plateauing within the first week in ampicillin treated mice (FIG. 9A). By contrast, C. albicans recovery in the feces and intestinal tissue was sporadic, and consistently at or below the limits of detection for control mice without drinking water antibiotic supplementation (FIGS. 9A and 9B). Importantly, despite this high density of intestinal C. albicans, no evidence of systemic fungal dissemination or other negative health consequence amongst colonized mice was observed. In particular, tissues commonly susceptible to invasive C. albicans infection, including the kidneys, liver, and brain, remained uniformly sterile (FIG. 9C), and C. albicans colonized compared with no antibiotic control mice gained weight at a comparable tempo (FIG. 9D). Thus, the human pathobiont, C. albicans, can achieve persistent long-term colonization in mice.

Using this model, how commensal colonization by pathobiont microbes impacts susceptibility to invasive systemic infection was evaluated. Given the lack of spontaneous disseminated infection in C. albicans colonized mice, susceptibility to invasive infection was probed by intravenous inoculation with the identical or marked isogenic virulent C. albicans strains. This analysis showed remarkably reduced susceptibility to systemic C. albicans invasive infection conferred by C. albicans intestinal colonization. Mice with commensal C. albicans showed significantly reduced mortality following intravenous infection with a lethal dosage of virulent C. albicans for immune competent mice on the C57BL/6 background (Jiang et al., Cell host & microbe 22:809-816 (2017)), and markedly reduced (>100-fold) fungal burden in the kidneys compared with control mice maintained on ampicillin supplemented drinking water without C. albicans colonization (FIG. 9E). To confirm that C. albicans in the target tissue of colonized mice directly reflects reduced susceptibility to intravenous infection, as opposed to dissemination from intestinal tissue, the advantages of an isogenic virulent C. albicans strain with GFP expression (Igyártó et al., Immunity 35:260-272 (2011)) were exploited, allowing oral inoculation and subsequent intravenous challenge using unique marked strains (FIG. 9F). Importantly, recoverable fungi in the kidney were uniformly from intravenous inoculation (GFP+), while fungi in feces were uniformly of commensal origin (GFP−) (FIG. 9F). Thus, despite retaining genetic virulence potential, pathobiont commensal C. albicans do not breech the intestinal barrier to seed systemic tissues. Together, these results show commensal C. albicans intestinal colonization efficiently protects against systemic invasive infection by the same pathobiont microbe.

Systemic Expansion of Protective IL-17 Producing CD4+ T Cells with Commensal C. albicans Specificity

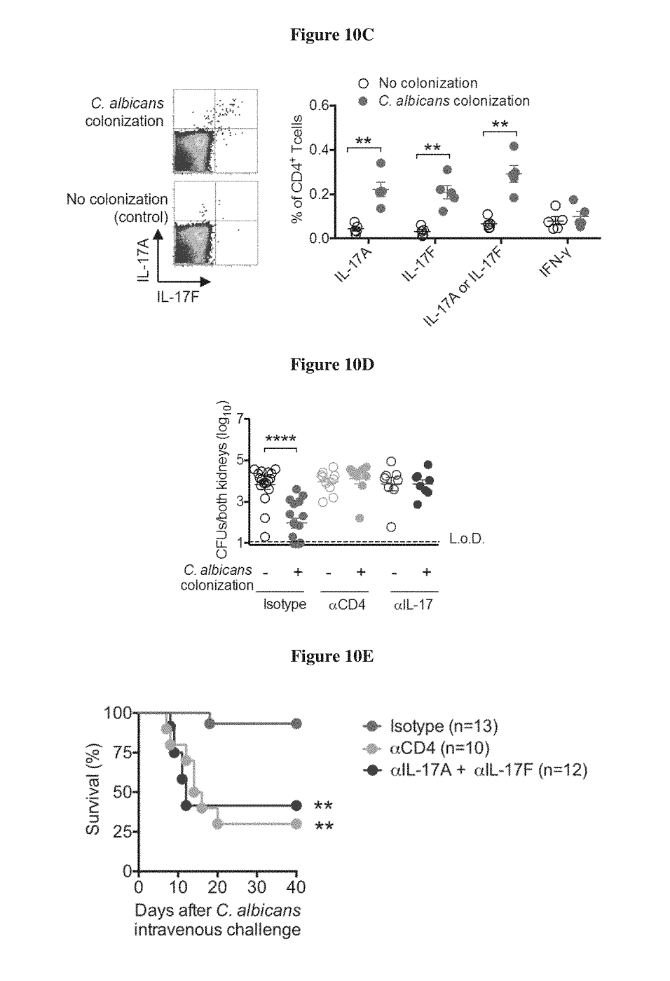

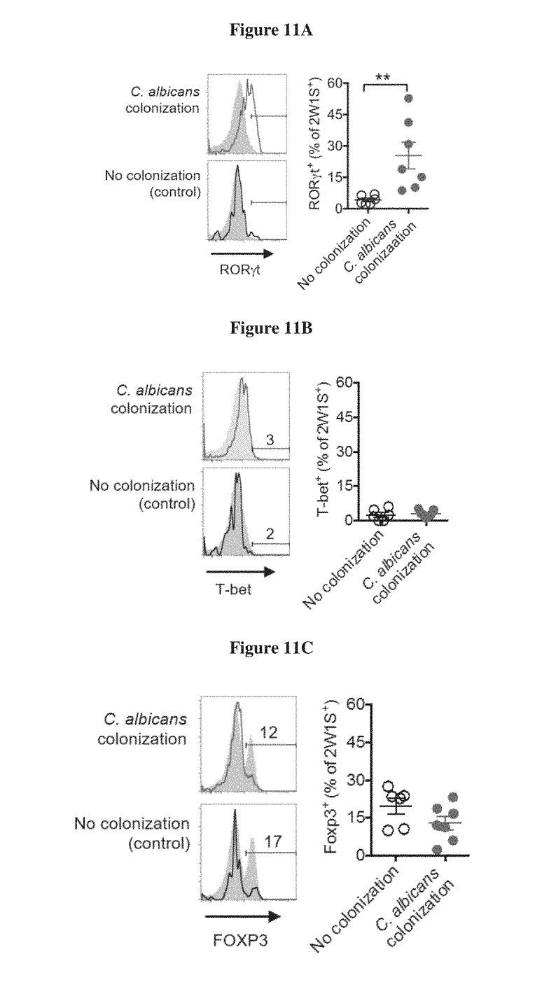

The immunological basis for protection against invasive C. albicans infection conferred by intestinal colonization was investigated by evaluating systemic accumulation of adaptive immune components with commensal C. albicans specificity. CD4⁺ T cells were identified as targets for the investigation since candidiasis is more prevalent amongst individuals with HIV infection or other immune comprising conditions with diminished CD4⁺ T cell function (Farah et al., Infection and immunity 70:724-731 (2002); Klein et al., New England Journal of Medicine 311:354-358 (1984)). To facilitate identification of CD4⁺ T cells with commensal C. albicans specificity, a recombinant virulent strain engineered to constitutively express the 2W1S55-68 variant of I-Ea peptide was used to establish intestinal colonization (Igyártó et al., Immunity 35:260-272 (2011)). In turn, the relatively high precursor frequency of endogenous CD4⁺ T cells with I-Ab:2W1S55-68 specificity allows cells with this surrogate commensal specificity to be precisely identified after staining and enrichment with I-Ab:2W1S55-68 MHC tetramer (Moon et al., Immunity 27:203-213 (2007)). This analysis showed robust expansion of CD4+ T cells with commensal C. albicans-2W1S specificity in systemic lymphoid tissue (spleen plus peripheral lymph nodes) within the first 10 days after colonization (FIG. 10A). Expanded CD4⁺ T cells with commensal C. albicans-2W1S specificity were almost all CD44^{hi} reflecting activation in response to stimulation by cognate antigen (Baaten et al., Frontiers in immunology 3:23 (2012)) (FIG. 10A).

To investigate how C. albicans intestinal colonization stimulates the differentiation of these systemically expanded cells, their expression of canonical T helper lineage defining transcriptional regulators was evaluated. Differentiation into RORγt expressing Th17 cells accounted for the largest subset of peripheral CD4⁺ T cells with commensal C. albicans-2W1S specificity compared with <5% among CD4⁺ T cells of the same specificity in no colonization control mice (FIG. 10B). Production of Th17 lineage defining cytokines IL-17A or IL-17F was also selectively increased amongst spleen and lymph node CD4⁺ T cells from colonized compared with control mice after stimulation in vitro with heat-killed C. albicans (FIG. 10C). Comparatively, the percent T-bet+ Th1 or FOXP3+ regulatory T cells remained similar amongst CD4+ T cells with commensal C. albicans-2W1S specificity in colonized compared with control mice (FIG. 11), and no differences in IFN-γ production were identified (FIG. 10C). Thus. C. albicans intestinal colonization preferentially primes systemic accumulation of fungal-specific IL-17 producing CD4⁺ T cells in mice similar to that described in the peripheral blood of healthy human volunteers (Acosta-Rodriguez et al., Nature immunology 8:639 (2007); Zhou et al., Immunology letters 118:72-81 (2008)).

To further investigate the necessity of these immune components in protection against systemic C. albicans invasive infection, the effects of their depletion initiated just prior to intravenous infection of resistant colonized mice was evaluated. It was that found administration of either CD4⁺ T cell depleting antibody or IL-17A plus IL-17F neutralizing antibodies each overturned the protective benefits of intestinal colonization, since fungal pathogen burden in the kidney and mortality each rebounded to levels comparable to control mice without C. albicans intestinal colonization (FIGS. 10D and 10E). Thus, C. albicans intestinal colonization primes systemic expansion of fungal-specific CD4 T cells and their production of IL-17, and each of these immune components are essential for colonization conferred protection against systemic C. albicans invasive infection.

IL-17 Responsiveness in Circulating Neutrophils is Increased with C. albicans Intestinal Colonization

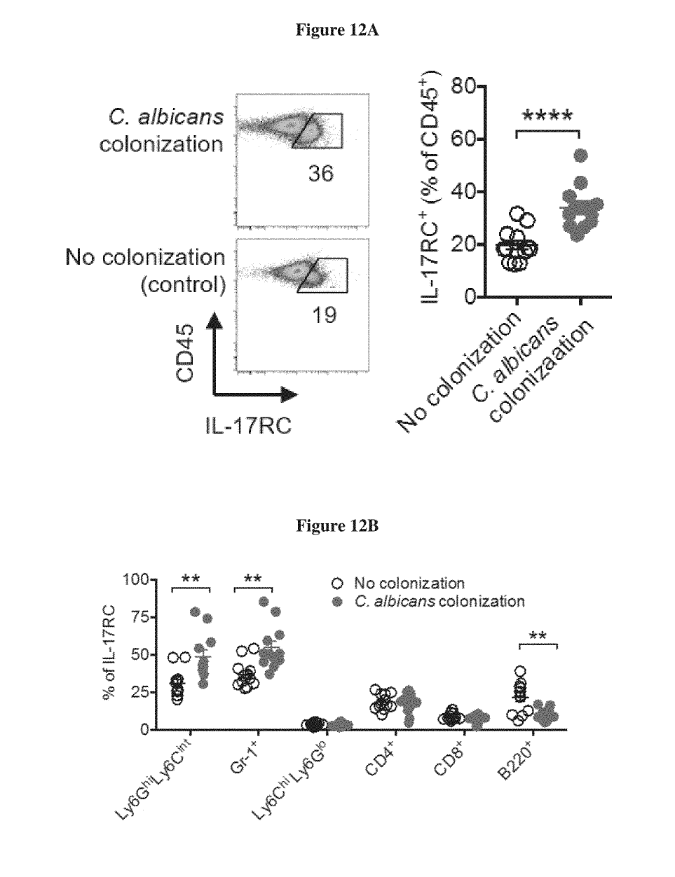

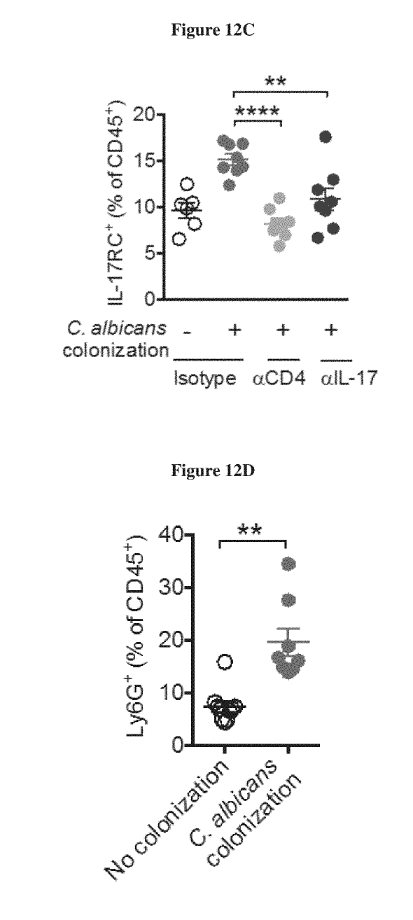

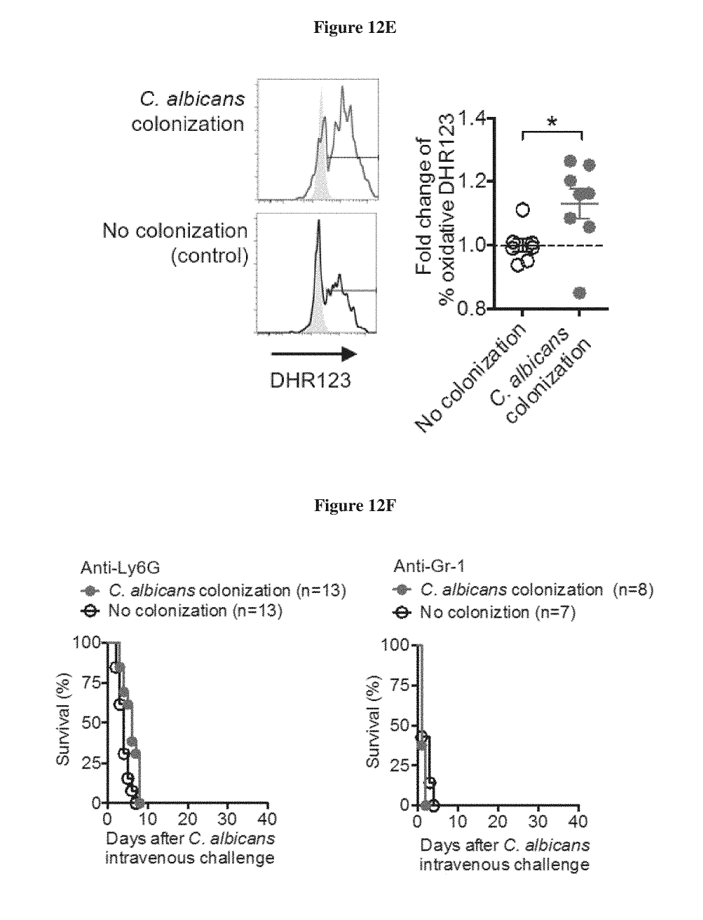

To investigate the IL-17 responsive cell subset(s) responsible for protection against systemic C. albicans invasive infection, shifts in expression of IL-17 receptor was compared amongst CD45⁺ leukocytes between mice with C. albicans intestinal colonization and no colonization controls. Rather than IL-17RA, which is ubiquitously expressed in leukocyte cells (Iwakura et al., Immunological reviews 226:57-79 (2008)), IL-17RC was targeted as an essential component of the IL-17 receptor complex whose expression is modulated in the context of inflammation (Taylor et al., Nature immunology 15:143 (2014)). Significantly increased frequency of IL-17RC expressing CD45⁺ leukocyte cells were found in the peripheral blood of C. albicans colonized compared to no colonization control mice (FIG. 12A). Amongst IL-17RC⁺ leukocytes, the proportion of Ly6GhiLy6Cint and Gr1⁺ neutrophils were each significantly increased, whereas no change (Ly6ChiLy6Glo monocytes and CD4+ or CD8+ T cells) or reciprocal reductions (B220+ B cells) were found for other leukocyte subsets (FIG. 12B). Enhanced IL-17 responsiveness by circulating leukocytes in C. albicans colonized mice was dependent on both CD4+ T cells and IL-17, since IL-17RC expression was reduced to levels comparable to no colonization control mice after administration of either CD4⁺ T cell depleting or IL-17A plus IL-17F neutralizing antibodies (FIG. 12C). Enhanced IL-17 responsiveness by neutrophils paralleled their expanded accumulation amongst circulating leukocytes (FIG. 12D), and activation shown by increased production of reactive oxygen species in response to C. albicans stimulation (FIG. 12E). Importantly, circulating neutrophils are essential for commensal C. albicans conferred protection, since depleting these cells using either anti-Ly6G or anti-Gr1 antibodies each overturned resistance against systemic C. albicans invasive infection, resulting in mortality comparable to no colonization control mice administered each depleting antibody (FIG. 12F). Collectively, these results show IL-17 and CD4+ T cells in mice with C. albicans intestinal colonization each continuously stimulate IL-17 responsiveness by neutrophils, which in turn, are essential for protection against invasive C. albicans infection.

Protection Against Systemic Infection Requires Persistent C. albicans Intestinal Colonization

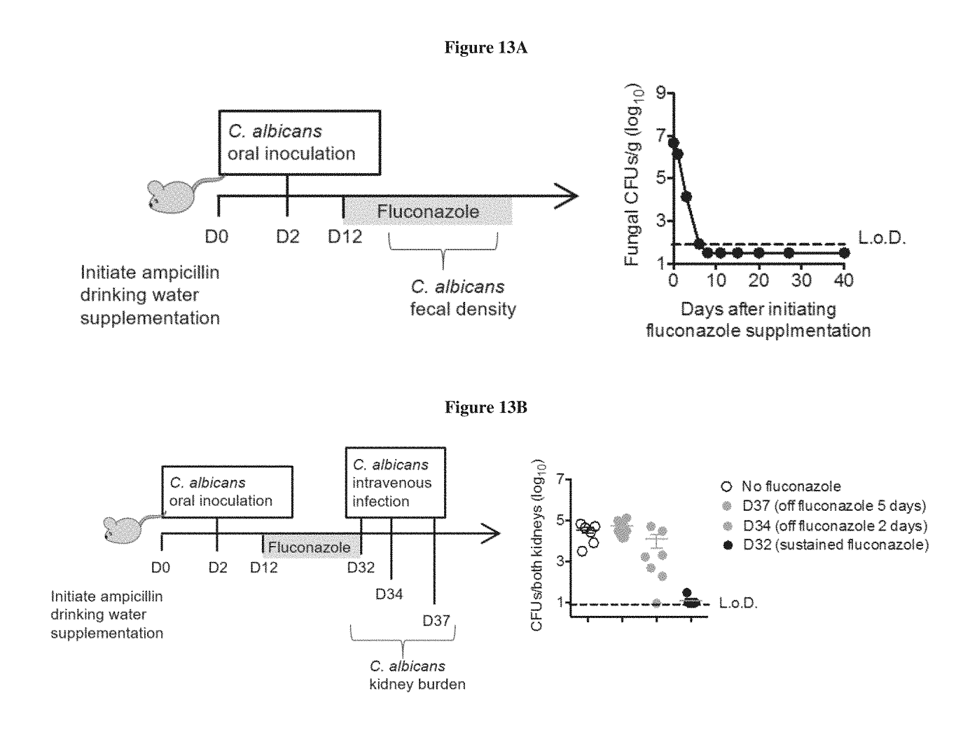

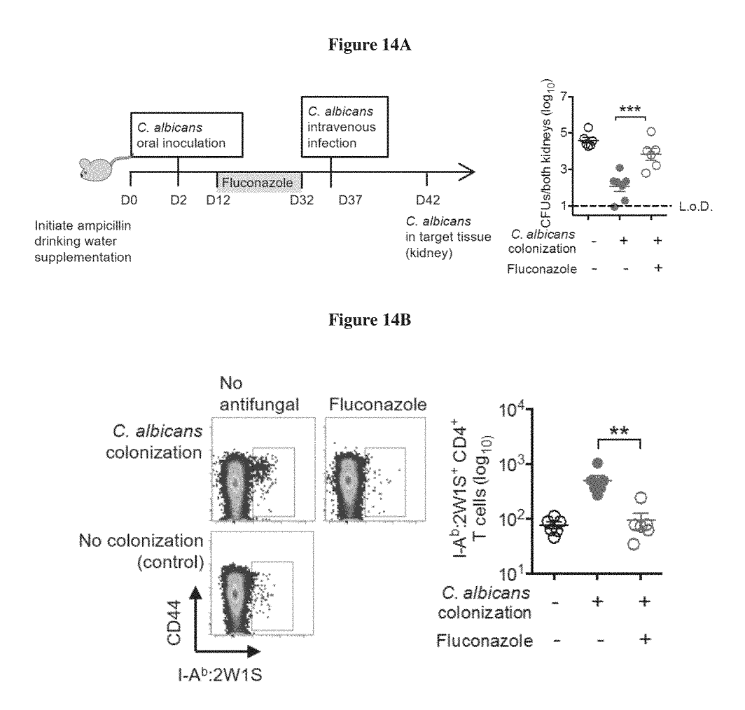

Shifts in the repertoire and density of pathobiont commensal microbes occur throughout development, and are intensified by changes in diet or the use of antimicrobial compounds (David et al., Nature 505:559 (2014); Lozupone et al., Nature 489:220 (2012)). Accordingly, the durability of commensal C. albicans conferred protection against systemic C. albicans infection was explored, in particular whether persistent C. albicans intestinal colonization is required. These experiments utilized fluconazole, an anti-mycotic agent, that when added to ampicillin supplemented drinking water of mice efficiently eliminates C. albicans intestinal colonization (FIG. 13A) (Iliev et al., Science 336:1314-1317 (2012)). However, since fluconazole would also artificially render resistance to systemic fungal infection at the time after removing fluconazole from the drinking water when susceptibility to intravenous C. albicans infection would be restored was investigated. These experiments showed that 5 days after discontinuation of fluconazole treatment, the fungal pathogen burden after intravenous C. albicans infection was restored to levels comparable to control mice without prior fluconazole treatment (FIG. 13B).

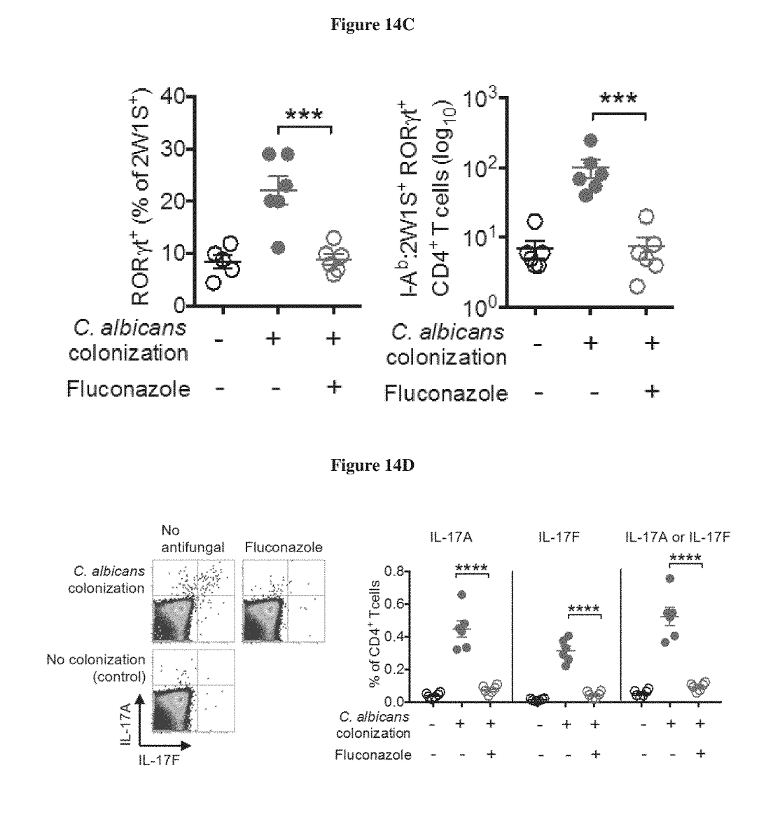

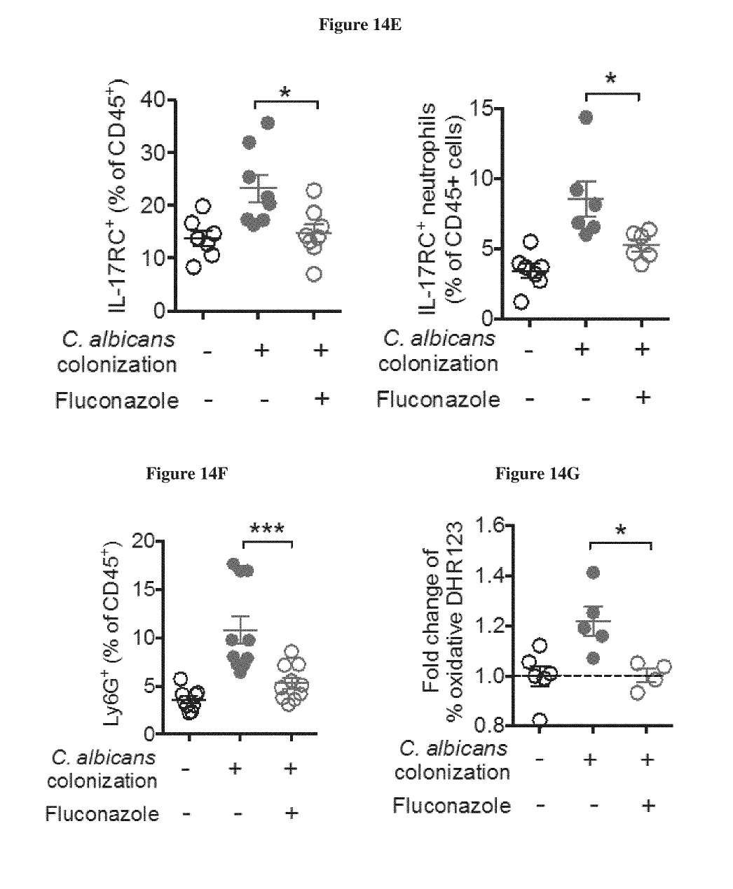

Using this approach, it was shown that protection against invasive infection and nearly all systemic immunological shifts primed by C. albicans intestinal colonization were eliminated with fluconazole. In particular, significantly increased fungal pathogen burden in the kidney was found after intravenous infection in fluconazole treated C. albicans colonized mice compared with mice with sustained C. albicans intestinal colonization (FIG. 14A). Reciprocally, the expanded number of CD4⁺ T cells with commensal C. albicans I-Ab:2W1S specificity, and their expression of RORγt were each reduced to background levels found in control mice without prior C. albicans intestinal colonization (FIG. 14B and FIG. 14C). Likewise, IL-17A and IL-17F production by spleen and lymph node CD4⁺ T cells in response to heat-killed C. albicans stimulation (FIG. 14D), IL-17RC expression by circulating leukocyte and neutrophils (FIG. 14E), expansion of circulating neutrophils (FIG. 14F) and their production of reactive oxygen species (FIG. 14G) were each similarly overturned for cells in fluconazole treated C. albicans colonized mice compared with mice with sustained intestinal colonization. Thus, persistent C. albicans intestinal colonization is required for sustaining systemic activation of fungal-specific Th17 immunity.

Commensal C. albicans Conferred Protection Extends to Other Extracellular Pathogens

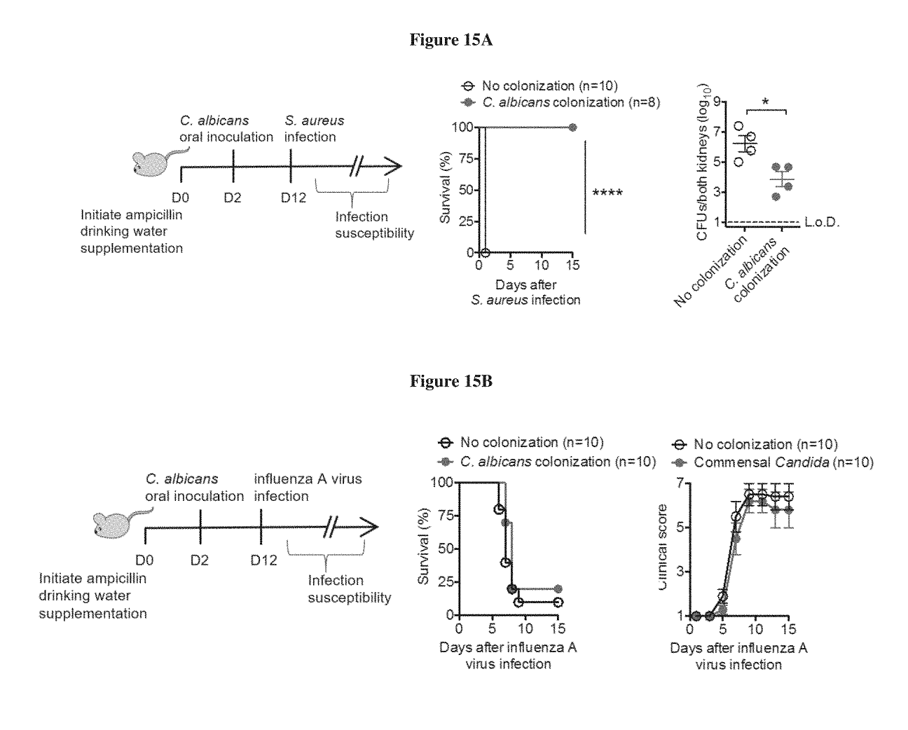

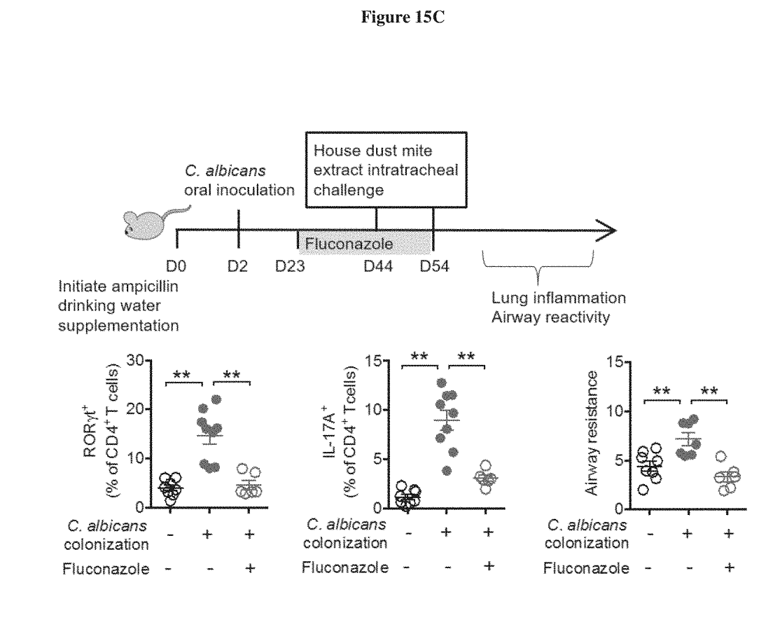

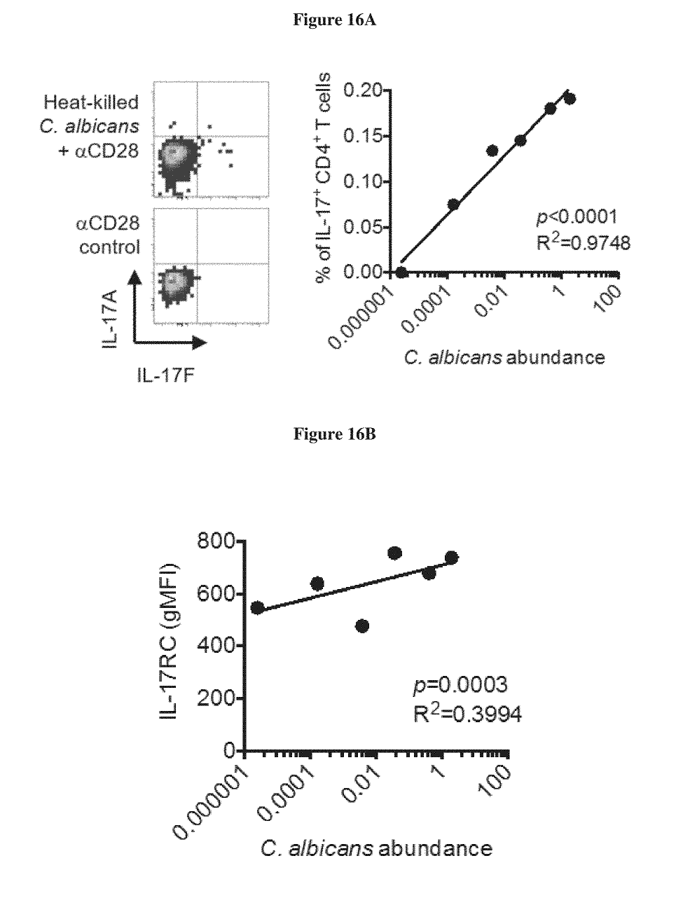

Given the expansion and activation of neutrophils by C. albicans intestinal colonization, together with the shared importance of these cells in protection against invasive infection by other pathogens (Kolaczkowska and Kubes, Nature Reviews Immunology 13:159 (2013)), the breadth of protection conferred by commensal C. albicans was ascertained. Since ampicillin supplementation in the drinking water is required for maintaining C. albicans intestinal colonization, microbial pathogens with natural or induced resistance to ampicillin were utilized for infection. Remarkably, these experiments showed C. albicans intestinal colonization confers sharply reduced susceptibility to systemic infection by the extracellular bacterial pathogen S. aureus. Mortality after infection with an inoculum of methicillin-resistant S. aureus that is lethal for ampicillin treated control mice was eliminated for mice with commensal C. albicans (FIG. 15A). Survival in mice with C. albicans intestinal colonization paralleled significantly reduced S. aureus bacterial burden in the kidneys after infection with a lower S. aureus inoculum (FIG. 15A). By contrast to this definitive protection against extracellular pathogens, no significant differences in survival, time to death or progression of clinical symptoms were found after infection with the intracellular viral pathogen, influenza A virus amongst mice with C. albicans intestinal colonization compared with no colonization controls (FIG. 15B). Together, these results show commensal C. albicans conferred protection is not restricted only to systemic C. albicans invasive infection, but extends to extracellular pathogens, such as S. aureus where neutrophils play a dominant role in protection (Rigby and DeLeo, Seminars in immunopathology (Springer), 2012).

Commensal C. albicans Activation of Systemic Th17 Inflammation Promotes Susceptibility to Airway Inflammation

Given these unambiguous important protective benefits against invasive infection, the potential for harmful consequences associated with systemic accumulation of activated neutrophils and Th17 immunity primed by C. albicans intestinal colonization was investigated. Allergic airway inflammation is increasingly recognized to be mediated by activated neutrophils and aberrant IL-17 production (Alcorn et al., Annual review of physiology 72, 495-516 (2010)). In turn, intestinal fungi have been shown to exacerbate asthma-like symptoms in antibiotic treated mice (Noverr et al., Infection and immunity 73:30-38 (2005); Skalski et al., PLoS pathogens 14:e1007260 (2018); Wheeler et al., Cell host & microbe 19, 865-873 (2016)). Accordingly, the potential for commensal C. albicans induced shifts in airway inflammation susceptibility was evaluated. It was found that the lungs of mice with C. albicans intestinal colonization compared with no colonization control mice contained sharply expanded levels of Th17 (RORγt+ and IL-17 producing) CD4+ T cells, and significantly increased airway hyperresponsiveness in response to intratracheal administration of the allergen, house dust mite (FIG. 15C). Importantly, and in agreement with the aforementioned experiments highlighting a necessity for persistent C. albicans intestinal colonization in sustaining systemic activation of Th17 immunity (FIG. 14A-14G), accumulation of RORγt+ and IL-17 producing CD4+ T cells in the lungs and increased airway responsiveness found in mice with commensal C. albicans were each efficiently overturned by eradicating intestinal colonization with fluconazole (FIG. 15C). Together, these results show C. albicans intestinal colonization primes systemic Th17 inflammation with broad immunological host impacts, on one hand protecting against systemic invasive infection by extracellular pathogens, but on the other hand promoting susceptibility to aberrant tissue inflammation.

C. albicans Fecal Colonization Density Positively Correlates with Systemic Levels of Fungal-Specific Th17 Inflammation

To investigate whether systemic Th17 inflammation driven by CD4⁺ T cell recognition of commensal C. albicans similarly occurs in humans, the relationship between fecal C. albicans colonization density, systemic expansion of IL-17 producing CD4⁺ T cells with fungal specificity and IL-17 responsiveness by circulating neutrophils was evaluated in intensive care unit patients naturally predisposed to have fungal colonization from antibiotic exposure. It was reasoned that comparing activation of systemic immune cells with C. albicans fecal colonization density at a single time point amongst individual patients would reveal a snapshot for how tonic C. albicans intestinal colonization impacts systemic immunological changes. To identify C. albicans-specific Th17 CD4⁺ T cells. IL-17A and/or IL-17F production by CD4⁺ T cells amongst peripheral blood mononuclear cells after stimulation with heat-killed C. albicans was evaluated. Both stimulation and no C. albicans stimulation control specimens were supplemented with anti-CD28 antibody to improve restimulation efficiency and cytokine production (Koehler et al., Frontiers in microbiology 9:1381 (2018); Liu et al., European journal of immunology 39:1472-1479 (2009)). The fecal abundance of C. albicans was evaluated by shotgun sequencing, followed by Kraken alignment of sequence reads against a custom comprehensive microbial genome database (Wood and Salzberg, Genome biology 15:R46 (2014)). Using this approach, highly significant positive correlations between the density of C. albicans colonization in the feces and proportion of peripheral IL-17 producing CD4⁺ T cells (p<0.0001; R2=0.97) (FIG. 16A), and IL-17RC expression by circulating CD15⁺CD16⁺ neutrophils (FIG. 16B) were identified. Together, these results indicate key components of systemic Th17 inflammation driven by persistent C. albicans intestinal colonization shown in mice are recapitulated in humans.

### Discussions

The intestine and other mucosal tissues harbor an extremely diverse and dynamic population of biologically active commensal microbes (David et al., Nature 505:559 (2014); Lozupone et al., Nature 489:220 (2012)). This microbial community encompasses many virulent pathobionts capable of invasive disseminated infection, including pervasive human pathogens such as Enterobacteriaceae, Staphylococcus, Streptococcus and Candida spp. (Brown et al., Science translational medicine 4:165rv 113-165rv 113 (2012); Canny and McCormick, Infection and immunity 76:3360-3373 (2008); Casadevall and Pirofski, Infection and immunity 68:6511-6518 (2000)). However, despite their exceptionally high colonization prevalence, systemic infection by these pathobionts that colonize mucosal tissues occurs relatively infrequently.

This immunological basis for this paradox was addressed in this study using an instructive murine model of long-term intestinal colonization with the human commensal-pathobiont C. albicans. It was shown that despite high density C. albicans intestinal colonization, spontaneous disseminated infection occurs very infrequently in mice, similar to human epidemiological data (Pfaller and Diekema, Clinical microbiology reviews 20:133-163 (2007)). Interestingly, resistance to systemic infection is not passive, but actively acquired and maintained by expanded CD4⁺ T cells that produce proinflammatory IL-17 cytokines in response to stimulation by commensal-pathobiont expressed antigens. In turn, IL-17 stimulates the accumulation, activation, and enhanced IL-17 responsiveness in circulating neutrophils that phagocytize invading fungi (Underhill and Pearlman, Immunity 43:845-858 (2015)). The protective benefits associated with systemic Th17 CD4⁺ T cell differentiation and activation of circulating neutrophils with C. albicans intestinal colonization further highlight why immune suppression and, in particular, neutropenia are dominant risk factors for candidemia in humans (Lau et al., Journal of clinical microbiology, JCM. 03239-03214 (2015); Patolia et al., British Journal of Medical Practitioners 6 (2013)). Importantly, the results obtained from this study demonstrated that these responses are similarly primed by C. albicans colonization in mice, and are essential for immunity against invasive infection provides an immunological mechanism that explains why invasive infection by commensal pathobiont microbes occurs so infrequently amongst immune competent individuals (Pfaller and Diekema, Clinical microbiology reviews 20:133-163 (2007)).

The discovery that commensal C. albicans primes systemic expansion of protective IL-17 producing CD4⁺ T cells with fungal specificity also provides important new clues for explaining discordance in the necessity of IL-17 in protection against C. albicans invasive infection in animals. In particular, while IL-17A and IL-17 receptor have each been reported to be indispensable for resistance against intravenous C. albicans infection using IL-17A- or IL-17 receptor-deficient mice (Huang et al., The Journal of infectious diseases 190:624-631 (2004); Saijo et al., Immunity 32:681-691 (2010); van de Veerdonk et al., Shock 34:407-411 (2010)), others have shown completely non-essential roles for these molecules using similar experimental tools and techniques (De Luca et al., Mucosal immunology 3:361 (2010)). Here, this study demonstrated that IL-17 neutralization and CD4⁺ T cell depletion each cause susceptibility to intravenous C. albicans infection only amongst mice with C. albicans intestinal colonization (FIG. 3D). These results suggest that the presence of commensal fungi dictates whether eliminating these immune components will negatively impact the susceptibility of mice to invasive infection.

Protection against C. albicans invasive infection has been widely used to show innate immune cells without antigen-specificity (e.g. monocyte, macrophage) can be trained to remember prior infectious encounters. Classical experiments show recent prior intravenous infection with a sublethal inoculum of C. albicans improves the survival of both wildtype and Rag1-deficient (deficient in both T and B cells) mice to intravenous C. albicans challenge (Quintin et al., Cell host & microbe 12:223-232 (2012)). More recently. Rag1-deficient mice were also used to show intestinal colonization with attenuated C. albicans mutant strains adapted for intestinal commensalism can prime protection against systemic virulent C. albicans challenge in the absence of adaptive immune components (Tso et al., Science 362:589-595 (2018)). The results of this study highlight the necessity for neutrophils (FIG. 4F), and additionally the absolute requirement for CD4⁺ T cells in protection against invasive C. albicans infection primed by intestinal colonization using virulent C. albicans (FIGS. 3D and 3E). In this study, long-term C. albicans intestinal colonization was established in adult mice using a single antibiotic, ampicillin (FIGS. 1A and 1B), compared with a broader cocktail (penicillin plus streptomycin) in the recent study (Tso et al., Science 362:589-595 (2018)). Likewise, addressing the necessity of CD4⁺ T cells by administering depleting antibody immediately prior to infection in the instant studies allows the importance of these cells in protection against systemic C. albicans invasive infection to be more definitively evaluated in isolation, bypassing potential variables associated with maturation and function of remaining innate immune cells in Rag1-deficient mice where T and B cells are completely absent throughout development (Karo et al., Cell 159:94-107 (2014); Quintin et al., European journal of immunology 44:2405-2414 (2014)).

Nonetheless, systemic expansion of IL-17 producing CD4⁺ T cells with commensal C. albicans specificity (FIGS. 3A and 3C), together with enhanced IL-17 responsiveness by circulating neutrophils which require ongoing exposure to IL-17 and CD4⁺ T cells in mice with C. albicans intestinal colonization (FIG. 4C), suggests that even innately trained non-antigen-specific immune cells can be further educated by adaptive immune components with commensal specificity. In this regard, durability is one potentially important distinction between non-antigen-specific immune cells trained by transient exposure to defined microbial compounds or acute infection conditions which have relatively short functional longevity (Quintin et al., Cell host & microbe 12:223-232 (2012); Tso et al., Science 362:589-595 (2018)), compared with the more sustained activation of neutrophils in mice with C. albicans intestinal colonization that we show relies on CD4⁺ T cell recognition of commensal C. albicans. However, durability in this context is also not absolute, since tonic stimulation by commensal C. albicans is needed for sustained activation and expansion of protective neutrophils and Th17 CD4⁺ T cells (FIG. 5). Further reinforcing the need for tonic commensal stimulation is the direct positive correlation between C. albicans density in the feces and levels of systemic C. albicans-specific Th17 CD4⁺ T cells or IL-17 responsiveness by circulating neutrophils (FIG. 7). Thus, an extraordinary fine-tuned dynamic interplay exists between commensal C. albicans and systemic immune cells that not only sense the absolute presence or absence of C. albicans colonization, but also transient shifts in colonization levels. These results open up exciting new opportunities to target fungi with either mycotic probiotics and/or antifungal agents for therapeutically fine tuning systemic immunity.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

What is claimed is:

1. A method for modulating immune responses in a subject, the method comprising administering to a subject in need thereof an effective amount of a composition comprising fungal cells or an extract thereof that contains one or more mannans.

2. The method of claim 1, wherein the subject is administered the fungal cells.

3. The method of claim 2, wherein the fungal cells colonize in the intestine of the subject after administration.

4. The method of claim 2, wherein the fungal cells are Candida albicans cells or Saccharomyces cerevisiae cells.

5. The method of claim 1, wherein the fungal cells are administered orally or intrarectally.

6. The method of claim 1, wherein the subject is administered the extract of fungal cells.

7. The method of claim 1, wherein the subject is a human subject.

8. The method of claim 7, wherein the human subject has or is at risk for intestinal mucosal tissue injury, intestinal infection, imbalanced intestinal microbiota, optionally wherein the human subject has a microbial infection by Streptococcous pneumoniae, Klebsiella pneumoniaie, Escherichia coli, Candida, or Aspergillus.

9. The method of claim 1, wherein the subject is on treatment of an antibiotic.

10. A method for modulating immune responses mediated by T helper 17 cells, the method comprising administering to a subject in need thereof (a) fungal cells or (b) an anti-fungus agent or a mycotic probiotic.

11. The method of claim 10, wherein the subject is administered the fungal cells, which colonize in the intestine of the subject.

12. The method of claim 11, wherein the fungal cells are Candida albicans cells.

13. The method of claim 11, wherein the subject is a human subject.

14. The method of claim 13, wherein the human subject has or is at risk for microbial infection, optionally wherein the microbial infection is caused by Streptococcous pneumoniae, Klebsiella pneumoniaie, Escherichia coli, Candida, or Aspergillus.

15. The method of claim 11, wherein the fungal cells are administered orally or intrarectally.

16. The method of claim 10, wherein the subject is administered with an anti-fungal agent or a mycotic probiotic.

17. The method of claim 16, wherein the subject is a human subject.

18. The method of claim 16, wherein the human subject has is at risk for an allergic airway disorder, an autoimmune disorder, optionally wherein the autoimmune disorder is an autoinflammatory disorder.

19. The method of claim 18, wherein the autoimmune disorder is rheumatoid arthritis, lupus, or multiple sclerosis.

20. The method of claim 18, wherein the autoinflammatory disorder is inflammatory bowel disease, psoriasis, or esophagitis.

21. The method of claim 16, wherein the anti-fungal agent is a polyene antifungal agent, an azole antifungal agent, an allylamine and morpholine antifungal agent, or an antimetabolite antifungal agent.

22. The method of claim 16, wherein the anti-fungal agent is administered to the subject orally.
